SEQUENCE LISTING (15 sequences)

SEQ ID NO: 1 - DNA, 16800 bp, Artificial Sequence
  Synthetic
ggtttacccgccaatatatcctgtcaagctcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtttttaaccatccttctactagacgtacactcgtacatcctctagtatttcctttatttatcttcccataagcccctgcctttaacgtttaactctcgctatcgacatggcattcttgaacgtttctgctgtccccagctgtgctttcgcacctgctttcgcaccccacgccggtgcaagccccattgtgcccgacagtttcccttgcgtaccccgttattcggacgacatatcccatttccgtttaacgttgtcgcttgacttctctgtcccgcgcccgctctttttgaacgcgcgtgtgcacttgagagcttctaccgacaaccccttaccatcgcttccccttggtttccacgctgaaacctttgttttggaattgaacggcagttcagctcccttctctataccgtcccgccacattgacttcgttgtgaaccggcctttctccgtgttccctactgaagtcctctccgtcagttcccttcgaacaccttctaggttgttcgctcttttatgcgactttttcctttactgttccaaaccaggtccttgcgttgaaattgcttctttttcaactccaccaccctgtttagtttcgaactgcgttgcacaaattcccacgcacgccgaaatggaaagtattcgttttccaacgaagactcttccagctggtcggctcttacagttccacaaacgaaaatacacgaaacgacctgaaactctaatcatacacgagagcggcttggctctcaaaaccagcgcgttaggtgttacatcgaaacctaattcgcgccctatcaccgtgaaatctgcttccggtgaaaaatatgaagcttacgagatctctcgtaaagacttcgaacgctcccgcaggaggcaacaaactcctcgcgttcgttctcacaagccccggaagataaataaagctgtcgaacctttcttctttccagaagaaccaaagaaagacaaacggaagagagcttctcttccgactaaagatgaaggtttcattacttttgggaccttgcgctttccgctttcggaaacccctaaggaagaacctcgtcttcctaaatttcgggaagttgaaattcctgtagtcaagaaacatgctgtaccagcagtagtttctaaaccagttcgaacgtttagaccggtcgctaccactggcgctgagtatgtcaacgctcggacccagtgttcgcgccgtccaagaaatcatccgattttacgcagtgcttcttatactttcggttttaagaaaatgccgctccagcgtttcatgaaagaaaagaaagattactacgtgaagcgctctaaggtcgtgagtagttgtagtgtaactaaaagtcctttggaagctttgaccagcatactaaagaacttaccacggtattcctacaactcagaacggttaaaattttacgatcattttatcggcgacgactttgaaattgaagtgcatccgctgcgaggcggcaaattaagtgtgcttcttattttgcctaagggcgaagcttactgcgtagtcactgcggccacgccgcagtaccacgctgctttaactattgcgcgtggcgatcgtcctcgcgttggtgaacttctgcagtaccgacccggcgaagggttatgttacctcgcccacgctgctctttgttgcgctcttcagaagcgcacctttcgcgaagaagacttcttcgttggtatgtacccgacgaagtttgtcttcgctaaacgactcactgaaaagttgggtcctagtgctctcaaacatcctgtgcgcggaagacaagtctctcgttcactcttccactgtgatgtggcttctgctttttcgagccccttttacagtttgccgcgtttcatcggcggcgtggaagaagaagctcctgagatcacgtcgtctcttaagcacaaggcgatcgaatcggtctacgaacgtgtcagcattcacaaagacaacttgttggctcgtagcgtggaaaaggatttgatcgactttaaagacgaaatcaagtcgctgtcgaaagaaaaacgttccgttaccgtccccttctatatgggggaggccgtacagagtggtttgacgcgcgcgtaccctcagtttaatttgagtttcactcacagtgtctattcggaccaccctgcggcggccggttcccgtctgttggaaaacgaaactttagcctcgatggctaaatcttccttttctgatatcgggggctgcccactttttcacataaagagggggagcacagattaccacgtgtgcagaccgatttacgacatgaaggacgctcaacgaagagtttcgagggaactccaagcgagagggctggtggaaaacctttcccgcgaacaacttgttgaagctcaggcgcgcgtttcggtgtgccctcacactctcggcaattgtaacgtgaaaagcgacgtgctaatcatggtgcaggtttacgacgcttctttgaacgaaattgcgtcggcgatggtcctaaaggaatcgaaagtcgcttacctcaccatggtgactccgggtgaacttctagatgaacgtgaagccttcgctatcgacgctttggggtgcgacgtcgtcgtggacacccgccgagacatggtgcagtacaaattcggctcatcttgttactgccacaaactgtcgaatataaagaatataatgttgactcccgcattcactttcagcgggaatcttttttcggtcgagatgtacgaaaaccgaatgggcgtcaactactacaaaatcactcgttcagcgtattctcctgaaatacgcggcgttaagacgctgcggtaccgccgagcctgcaccgaggtcgtgcaagttaaactgccacgcttcgataaaactctaaagacgtttctctccgggtatgattacatatatttggatgcgaaattcgtctctcgagtcttcgattacgtggttagcaactgtagcgttgttaacagtaaaaccttcgagtgggtgtggagttacataaaatcaagtaaatcgcgcgttgtcataagtgggaaagtgatacatcgagatgtgcatatcgacctcaaacactctgaatgttttgcggctgtgatgctcgcggttggggtgcgttctcgcacaactactgaatttttagcgaagaacctcaattattacactggcgatgcttcgtgctttgagactatccgtttcctctttcgggagtggagccggagggcttacgcggaaataaatcgcagttttcgaaagcttatgaagagcattctttccgctgggttagattacgaatttctcgatcttgacaactcgctccaacacttgcttgaatactcagaggttgaagtgcgcgtttcaatcgctcagaatggtgaggtggattgcaacgaagagaaccgtgttttgacggagataatagccgaagcagcggacaggaaatccatcgctcaaggtctcagcggagcgttgagctccgttccaacgcaaccgagaggcggtctgagagggggtagtcgccgaagtggagtttttttcctttataacttggtagaggaagtcggaaatcttttcttttccgtcggtgatgccgtgcggtttcttgttaagttatttaaaactttttccgactctcccatctttcgggtcgttcggatgttcctggacttggcggaagcagcttcaccgttcgtttcggttgtttctttgtgcgcgtggttgcgcgaagctgtaagcgctttctcaggctgggttgccgacagaacggtgtcggaaagtgtaaaaacctttgtaaaccgtacggtaaaaaggtttctaaactttatgtctgcaaaaaccctgacgaaaaaattttttaggtttttcttatccgcttccgccttagctaaaaccgttgtgaggaaggcgaaggtgattttggaagcttactgggaagtgtggttcgaatcgattctttcggatagtggagagtatagtgctgttgaattttgtagcagcgttgtgatcacactgttaacgaattccggtcggctgctacctgggtttagtccatccgctgtcattactgaagtactgcttgacctagccacgaaaatatccattgaagttcttctaaaacaaatttctcctgtcgactcgacagcttcttcggccttatatcgccgcgtcctaagcgagattctgtcgaattttcgcacaatgggcgaacacgggatcttcactaaagtctttttactctgcgggtttctacccgtgtttgttaggaagtgcgtcgctctgtgcgttcctggtgacatggcgacgtacgcccggtttctcgaatacggggtcgacgatctcttctttttgggaaggtccgtgaactcgattaagaactacctatgtgtggttgctgccgggttggtggactccattgtcgattccgttgttttgaaactttccggtgtcgccaaagagcgagtgcttggttttaaatcaaaaatcataaaaaattttttaaacgtctttaggaaggcgaaagtcgttacacgtacttcttccagcacggacttgtcggaagacgaatatttttcgtgcgacgaaagcaaacccggcttgagaggcggttcctctaggttcacgctttcgcgattgcttgacatctttttcaatttcctgaaaagttcaaagctcgtcatagagaatgcgtgcttttctgcttacgaaaggattgaaaggaacatgaaactatacttttttcctttaaattcttcggaagaagaagctcgccgcttaattcggtgcgcgggagacttcgactacttaagcgatagcgctttcgacgaagatgaaatgttacggcaagctttcgaacaatactattccagtgacgacgagagtgtaacttacgatggaaaacccacagttctccgtagttacttgaacgtgagtagaaggttcttggaaacgttctgtaacggcccgaagtttttcgtcaaagtttcgaattactttaaagcgctgtatagtcgtttgctccgtgttctaccgtgggtagacagaaatctttctgactcgccaggacttaaaggaggtaacgagaaagctcttcttgcgaagttccttaaaacctgcgtcataaccgcttgcgaatgtgtgtcgcagatatgctgcctgcgtcttattcgcttatgttgggggacaccagcgtgtggtttagttaggttattttacataacttattccggtactcgcgtgttgtcgcgtgttgtggtcgcggtggctgtatgccctcttttggtcagaaacgaactggatggcttgagtgatggattaaccaacatgggcgtttcggtttttcgtcgcttgttcgtcgcccttcggcgcgcgctttcggcgtactctaattccgccctacgacggaagattttcgaattcattttcggaaacattcaccacccttttgatgtcgcagtaatcgaaacaaacgaagttgcgccggaacctctttcaccggaagtggacattgatgtcgactgcgactttggttccgattcagaatcggtttcttcagatgaagtcgcgtcgattccccgtccaggcttacacggtgggaataggcgctcttccaactttctaacctctctcgtgaaggttgtttttaagttggctgggcgcattccgcgtttgcttttccgtttacgcaattttgtggcgtactttgttgaacgacggctggcttcaaaaaggttgaagacgttcatcggtctggccaggctgtttgataatttctcactcacttcggtagtttaccttctccaagagtacgattcagtgctgaacgcatttatagacgttgagctagtcctacttaactcgggtagcgtgaatgtgctacctttagtttcttgggtcaggggatctctaacgaagttagcggaagtgatcgttggttcaggcttcgcttcgtttctaggaaggatgtgctgtcgtgtgtccgactggtgctcctcgtcttcgaacgccggttgtaactttatgagtccggttcgtacgaaagggaagttcgttcccccttcgtcttccggttcaaccgcttcgatgtatgaacgtctcgaagctctcgagagcgatatccgcgaacacgtgctttccacgtgtcgtgtaggaagcgacgaagaagaggaaaggccgaaagaagtgacagaaccaggaattgaacatacttctgaagatgttgttcccattcgttcacactcgcaacccttatctggaggtgaatgttcgtattctgaagatcgtgaagagaatgaacgagcgaacctgttaccgcacgttagcaaaatcgtcagcgaacgaaggggtttggagaccgcccgtcgaaacaaacgtactctacgtggtgtaagcgagtttctcaacgctattaatactagcaatgagcaacctaggccgataatcgttgaccactctcctgaatctcgcgcgttgaccaactccgtgagggaattctattacctccaggaactcgctctttttgagttgagttgcaaacttcgtgagtattacgatcaattgaaggttgcgaattttaacagacaagagtgtttgtgcgacaaagacgaagacatgtttgttctacgagccggacaaggtgtagtttccggcagaaactcgaggttgcctcttaagcatttcaaggatcacgaattttgttttcgctctggagggttggtcccttacgacggtaccagcagagtggacaccatttttcacacgcaaacgaatttcgtttccgcgaacgcgcttctttcgggctatctctcctatagaactttcactttcactaatttgagcgctaacgtactgctgtacgaagctcctccaggtggtggaaagaccacgactttgataaaggttttttgtgaaactttttcaaaagttaattcgctgattttaacggcgaataagagttcgcgagaagaaatacttgcgaaggtgaatcgcatcgtacttgacgaaggcgatacgcctcttcagacgcgtgacaggattttaactatcgattcttatctaatgaacaacagaggtttgacgtgtaaggttttgtacctcgatgagtgtttcatggttcacgctggagctgctgtagcttgcatcgaattcaccaaatgcgattcagccatcttatttggagacagcaggcaaattcactacatagaccgtaacgaattggatactgctgttctttcagatttgaaccgttttgtcgatgatgaatcgagagtatatggtgaagtctcatacaggtgtccttgggacgtttgtgcttggttgtcaactttctacccgaaaactgtggccactaccaacttggtttcagccggtcaatcttcgatgcaagtacgcgagattgaaagcgtagacgacgtcgaatattccagtgaattcgtctacttgactatgttacagtcagagaagaaagatctgctgaaatctttcggcaagaggtctcgttcgagcgttgaaaaacccacggtcttaacagtccatgaagctcaaggtgaaacctaccgcaaagttaacctcgtcagaacgaaatttcaagaggacgatccttttcgtagcgagaaccacatcacagtggccttgtctaggcatgtcgagagtctgacctattcggtcctgagtagtaaacgtgacgacgcaatagctcaagctatagcgaaggcgaaacaacttgtggatgcctatcgcgtttaccccacgtcatttggtgggagtactcttgatattagtgttaacccttctacatctgacaggagcaaatgtaaagcttcctctgccccttacgaagttataaacagcttcttggagagcgtggttccgggcactacttcagtagactttggggacgtttccgaagagatgggcactcaggtttttgagtccggtgctgataacgttgttattcgtgattccgcacctgttaacaagtcgacggatcacgacccgcagcgggtttagctcgattcgctcgcaggcgattcctaagaggaaaccgtcgctgcaagagaatttatactcttatgagtcgcgtaattacaactttaccgtttgtgaacgtttttccggaccgcaggagttcggacaggcgatggcgatggttatgttggaacgaagctttgacttagagaaagttgctaaagttagaagcgatgtgatcgccataacagaaaaaggggtgcgaacatggatgtcaaaacgtgaaccttctcagcttagggctcttagtagtgacttacaaaagcctctaaacttggaagaggaaataacgacttttaagttgatggttaagcgggacgcgaaagtcaaactcgattcgtcgtgtttggtgaaacacccaccagcgcagaatataatgttccatcgcaaggcggtgaacgcgattttctcgccgtgtttcgacgagtttaaaaatagagtcattacctgtacgaattcaaatattgttttctttaccgaaatgactaactctactctcgcgtcgatagcgaaagagatgctggggagcgaacacgtttacaacgttggggaaatagacttttcgaaattcgacaaatcccaagacgctttcattaagtcgtttgaacgaaccttgtattcagcgtttggtttcgacgaagacctgcttgacgtgtggatgcaaggtgaatacaccagcaacgctacaactctggacggtcaactttctttttccgtcgacaaccagaggaaatcgggcgcttctaatacgtggattggtaattccatcgtgactcttggcattttgagcatgttctattacaccaatcgatttaaggctcttttcgtgtccggggacgactctttgattttctccgaatctcctattagaaattcagccgatgcgatgtgcacagaactcggttttgagactaagtttctcactccgagcgtcccgtatttctgctcaaagtttttcgttatgaccggtcacgacgttttcttcgtgcccgacccttataaacttttagtgaaattaggagcttctaaggatgaagtggacgatgagtttctgtttgaagtgttcacctcttttcgcgatttaacgaaagatttagtcgatgaaagagtgatcgaactcttgacgcatttggttcacagtaagtacgggtacgaaagtggtgatacgtacgccgccctgtgtgctattcattgtattcgttcaaacttttcatcgttcaagaaattgtaccctaaagttaagggctgggtcgttcactacggtaaactgaagtttgtgctgcgcaaattcgcgaactgttttcgcgagaagtttgacactgctttcggcgaagcgtactttcttacttacgacgaaacttgagactgtgtggtaactcggttgttgttttgtttgttcgtgtgtctgtagtcctacttcgcggtgatggactgtgtactccgctcgtatttattactcgcattcgggtttttgatttgcttgtttcttttctgcttagtggtttttatttggttcgtgtataaacaaatactttttcgaaacaccccgccttcgaacgaagcgcgtttcaaccgttccacggtcgtgtgatggttgttttcggattagatttcggcactactttctcgagcgtttgcgcttatgttggagaagaactctatttgttcaaacaaagagatagtgcttacatacccacttttgtgtttttacattccgatactcaagaagtggctttcggttacgatgccgaagttctttctaacgacccttcggttcgtggaggtttctatcgcgacttaaaacgctggataggatgtgacgaagagaattacggggattacctggaaaaactcaagccgcactacaagacggaattgcttaaagttgcgcaaagttcgaaatccaccgtgaggttagactgttactcaggtaccgtgccgcagaacgccactctaccgggtttgatagcgactttcgtcaaagctctcatttccacagccagtgaagctttcaagtgtcagtgcaccggcgttatttgttcggtccctgccaactataactgtctgcaaaggtcttttaccgaaagctgcgtcaacctaagcggctacccctgtgtttacatggttaacgaaccgtcggctgcggcgctctcagcttgttccagaatcaaaggcgctacttcgcccgttctcgtgtatgatttcgggggtggaacgtttgatgtttccgtgatttcagcgctcaacaacacttttgtggtgcgcgcttctgggggcgacatgaatttgggaggtcgtgatatcgataaggcttttgttgaacacttgtacaaaaaggctcaactgcccgtgaactacaaaattgacatttccttcttgaaagaatcgctctcaaagaaggtttccttcttaaacttcccggtggtcagtgaacagaacgtaaaagttgacgttttggttaatgtcagcgaactggctgaagtggcggctcctttcgtagaaagaacgatcaagatcgttaaagaagtttacgaaaaatatcgtggtagcatgcgattggaaccgagtgttaaagctaaactgcttatggtaggtggttcttcctacttgccaggtcttttatcgcgtctttcctccgtgcctttcgtggaagaatgccttgtgctgcccgatgctcgagcggcggttgccggggggtgtgctttgtactcagcttgtcttcgaaacgattctccgatgttgctggtcgactgtgcagctcacaatcttagcattagtagcaaatattgcgaatccatcgtttgcgttccggctggttcacccatcccgtttacgggagttcgaacagttaacatggctggttccaacgcttccgctgtttacagtgcggcgcttttcgaaggagacttcgtcaaatgtcgcttaaacaaaaggattttctctggtgacgtcgccttaggagacgtcggggtgttcggttcaaacactaggacggtcccgttaacactcgaaattaatgtttcgagcgtcggaacaatcacgttttccctcgtcggaccgacaggtgttaagaagttggtgggtggaaatgctgcttacgatttctcgagttatcagctcggggagcgcgtcgtcgcagacttgcacaaacataattctgataaagtgaaacttattcatgcgttgacgtataagccttttcaaaggaagaaattgactgacagcgacaaagctttgttcttaaagagactaagtgcagactaccgacgtgaagcgggaaagttcagttcatatgacgacgcggttctcaactccagcgaactactactggggagagttattccgaagattcttcgggggtcgagagtggaaaaacttgatgtctgaagctgcttcggttaggcggcctgtgtatgatcgcgactttcgtttttcaaacggtgaagtattaagtcgcaagaatttcagcgactcaaccggcgaatcatttgtgcgcgagttttcattgctgctgactttcccaaagacttatgaagtttgcaagttatgcggtgtggctatggaacaagctctgagcggcatgaatcgtctatccgattataacgtctccgaacttaacgtcgtcgacgtgaaaaccgtgggttgcaaatttaatatacaaaccgtcactgaattcgtgagaaagctcaacgggaatctggccgaaccttctctcgtcgagcactgttggtctctgtctaattcttgcggcgaactgatcaatccgaaagacacgaagcggttcgtttccctcatctttaaagggaaggacgtcgtcgaaagcaccgacgaagccgtagtttcctcttcttacttagattacctttcccactgtttgaacttgtacgaaacgtgtaatctttcgtctaactcagggaagaaagctttgtacgacgaatttctaaagtacgtgatcgtttatcttgaaactagtgacttagaataccgttcactttccgacaaccctttggttgccggtgttctttacgatatgtgttttgaatataacacgctcaagtcgacgtacctaaagaacatagagtcattcgattgttttttgagtctgtacttaccgttgctcagtgaaatcttttcgatgaattgggaacaaccagcgcctgacgtccggttgcttttcgaactcgatacaaccgaacttcttttgaaaattccgactatcaacacgcacgactcaacttttttgtataagaataagttgaggtatctggaatcctactttgaagacgactctaatgagctcataaaggtgaaagttgattcactcttaacccgagacaatcccgaactgaaactagctcagaggtgggtaggtttccattgttactacggggtttttaggaccgctcagactaggaaagtgaaaagagatgcggagtacaaactgcctccagctctcggtgaattcacaataaacatgagtggagtggaagaattctttgatgagctgcagaagaaaatgccttccgtttctgttcgtcgcagattttgtggcagtttgtctcacgaagctttttccattttcaagagattcggggtgggttttcctccgatcactagattaaacgttcccgtgaaatactcctatctaaacgttgactattacagacacgtgaagagagcgggcttgacgcaggacgagttgactattctcagtaacatcgaattcgacgtcgctgaaatgtgctgcgagagggaagtggctttacaagcccgtcgcgctcagcgcggcgaaaaaccatttcagggttggaaaggtgttaaaaacgaagtttctccgcacgcgcgttcctcaattcgagtgaaaaagagcaacgaatcgttactaaacgttttgtggaaagatgttggcgcccgaaggcaggggagacttaatccacttcaccgaaaacactagagatgccatggaaactttcttcaacagctacgatttagctgaatactccgaagtgaaccctaacaaacttaatcggaaagagaccgacgagttgttaggtgttattcgagagagatttaaatccgaattggtgataacagatgaagattttgtgaaacatttagccttcgcgttgattcgcgcggccaatatcaccactagtacgaaagtcaactacgtcggagcgtacgagtatacgatagggggaaagaagttcctggtcaaggacgcttgggttttccctttgataaaggagtgtatgaaaaagttcaacaaacccaatcctgtgaggacgttttgtgctactttcgaagacgcttacatagtgatagctcgttcgttgcctaaactgttcctgaatagaaccattggcaaacgtgggatcccgtcagggtatgagttcctcggggcagattttctaactgcgaccagcgtgtgtttgaacgatcacgaaaaagctatcgtactacaggcctcaagagctgccattgatagagcagtctcttcgtcggtcgacgggaagatcgtcagtcttttcgacctcggtcgtcttagttaacacagttactaaggttccattttattattgcattgtttttcatttagtgtaatcgtacttgagttaattaaggcgcgcctgtacagctagctatggccggcctcaacgtgtccgttaatgtccgaccacgatcgtgctgtagctttgagtgcatctaggaacgcactcgaccgatcggcggcgtctcaaattgataaaaagatggttagtttgtacgacttcggtaaggtagtgtatacctaacagcttgcttctacctgacacagttaagaagcggcataaatcgaagccaaaccctaaattttgcaactcgatcaattgtaacctagagcgaagtgcaatcaatgggatcagccgaacctataagtgcaatcgcgacttttgaaaacgtgagtctcgcagaccaaacgtgtttgcacggtgaagactgcgacaaactacggagggatttcgaagagtgtttgaaattgaaaggggttccggaagataaactcggtctcgcgttaggactttgtttgtactcctgtgcgacgataggtacttctaataaagttagtgtccaaccgacgtctactttcatcaaagcttcgttcggtagtgggaaggaattgttcctcactcacggtgaactgaggtcctttctggactctcagaaacttttagagggaaagcctaacaaattgcggtgtttctgccgcacttttcagaaggattacatatccttcgcgaaggaataccgaggaagactgcctccgattgctagagccaaccgtcacggtctacctgctgaagatcactacttagctgctgatttcatatcgacatcaacagaacttactgacctacaacaaggtcgtctgctgttggcgcgcgaaaacgccactcacacagaattctcgtctgaatcaccagtaactagtttgaaacagctgggtcgtggtctagccaccggaaaatgactagctctgtcgaactagctcagacgaaacccctttttagagtgttgctattaaagggtttcgttttttatattgttgcaatcgaaacagaagaagaatcgcctgaagttgaacttcctttggtttacctccacgatctcgagttgaatgttaacaaaaaggggaagatcgaatcttcgtacatcgactttaaatcttgtatgactaggttgaaacccagttcggtctcttacactcgagtgagttctgggaaaccttcagaagatttctcttcgtcatactcaggaaaaactttcgattcaaaaatacttaaccgtaaagtgacgtttacattcgaaagtggcattcaactcgtgttcgggatgtacggtcgcgatcagcggtgtgtctcttccgaatacttgtggtttgaaaacgtattcgtcggcgcgcactgcggcaccttaccctattgtctgaattgcgaattagacaaaagcggtggtgaattggaaattttgactttttcaaagaatgaagtttttctttaatgactgtgaaacttctcgagccatatctaggtctgaatcgctcctacgtcgcgtgaaggaacttggaacgaactcgccgcaaagcgaggtctctgagtgcatcaacgaattcaacgaactagcaaggtttaatcacttgttagttacggtcgaacacagggaacggatggaaaaacatccgaaacagtcgtcggaacttcgaactccatcacgacttggcgaaatgctcaaagaaattcgtgcgttccttaaggtgcgcgtggtaactcctatgcataaggagactgccagcgaaactctgaacgcgtttctggaagagtactgccggattactggactcactcgcgaggatgccttgcgagaaaaaatgaggaaagtaaggagtacggtgctgtttcatcactcggaacttttgaagttcgaagtgactgaaaacatgttcagttttactgaactactaaaactaaatctaagcttgagggtgatttcttctcaaatcctcggtatagctgtatagagaagtgacggaggtgacctacctgccgtgtaatttaagtcgtcacagagtgacaacggcaccaagtggtgctttgtgcgtatgtaaattgcgaagtgaaaaaattttttaaaaaaatttttcacactcctagcgaagtcccgctaggaagaaaaaataagggccgtcaagacatgattcacatgtcttctgatgagtccgtgaggacgaaacggcccggggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacagaatatagcgcgcaaactaggataaattatcgcgcgcggtgtcatctatgttactagatcaagcttatcgataccgtcgacctcgagggggggcccggtaccaaaaccaccccagtacattaaaaacgtccgcaatgtgttattaagttgtctaagcgtcaatttgtttacaccacaatatatcctgcca

SEQ ID NO: 2 - DNA, 10933 bp, Artificial Sequence
  Synthetic
ggtttacccgccaatatatcctgtcaaacactgatagtttaaactgaaggcgggaaacgacaatctgagctcgcgtgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagcagtttttaaccatccttctactagacgtacactcgtacatcctctagtatttcctttatttatcttcccataagcccctgcctttaacgtttaactctcgctatcgacatggcattcttgaacgtttctgctgtccccagctgtgctttcgcacctgctttcgcaccccacgccggtgcaagccccattgtgcccgacagtttcccttgcgtaccccgttattcggacgacatatcccatttccgtttaacgttgtcgcttgacttctctgtcccgcgcccgctctttttgaacgcgcgtgtgcacttgagagcttctaccgacaaccccttaccatcgcttccccttggtttccacgctgaaacctttgttttggaattgaacggcagttcagctcccttctctataccgtcccgccacattgacttcgttgtgaaccggcctttctccgtgttccctactgaagtcctctccgtcagttcccttcgaacaccttctaggttgttcgctcttttatgcgactttttcctttactgttccaaaccaggtccttgcgttgaaattgcttctttttcaactccaccaccctgtttagtttcgaactgcgttgcacaaattcccacgcacgccgaaatggaaagtattcgttttccaacgaagactcttccagctggtcggctcttacagttccacaaacgaaaatacacgaaacgacctgaaactctaatcatacacgagagcggcttggctctcaaaaccagcgcgttaggtgttacatcgaaacctaattcgcgccctatcaccgtgaaatctgcttccggtgaaaaatatgaagcttacgagatctctcgtaaagacttcgaacgctcccgcaggaggcaacaaactcctcgcgttcgttctcacaagccccggaagataaataaagctgtcgaacctttcttctttccagaagaaccaaagaaagacaaacggaagagagcttctcttccgactaaagatgaaggtttcattacttttgggaccttgcgctttccgctttcggaaacccctaaggaagaacctcgtcttcctaaatttcgggaagttgaaattcctgtagtcaagaaacatgctgtaccagcagtagtttctaaaccagttcgaacgtttagaccggtcgctaccactggcgctgagtatgtcaacgctcggacccagtgttcgcgccgtccaagaaatcatccgattttacgcagtgcttcttatactttcggttttaagaaaatgccgctccagcgtttcatgaaagaaaagaaagattactacgtgaagcgctctaaggtcgtgagtagttgtagtgtaactaaaagtcctttggaagctttgaccagcatactaaagaacttaccacggtattcctacaactcagaacggttaaaattttacgatcattttatcggcgacgactttgaaattgaagtgcatccgctgcgaggcggcaaattaagtgtgcttcttattttgcctaagggcgaagcttactgcgtagtcactgcggccacgccgcagtaccacgctgctttaactattgcgcgtggcgatcgtcctcgcgttggtgaacttctgcagtaccgacccggcgaagggttatgttacctcgcccacgctgctctttgttgcgctcttcagaagcgcacctttcgcgaagaagacttcttcgttggtatgtacccgacgaagtttgtcttcgctaaacgactcactgaaaagttgggtcctagtgctctcaaacatcctgtgcgcggaagacaagtctctcgttcactcttccactgtgatgtggcttctgctttttcgagccccttttacagtttgccgcgtttcatcggcggcgtggaagaagaagctcctgagatcacgtcgtctcttaagcacaaggcgatcgaatcggtctacgaacgtgtcagcattcacaaagacaacttgttggctcgtagcgtggaaaaggatttgatcgactttaaagacgaaatcaagtcgctgtcgaaagaaaaacgttccgttaccgtccccttctatatgggggaggccgtacagagtggtttgacgcgcgcgtaccctcagtttaatttgagtttcactcacagtgtctattcggaccaccctgcggcggccggttcccgtctgttggaaaacgaaactttagcctcgatggctaaatcttccttttctgatatcgggggctgcccactttttcacataaagagggggagcacagattaccacgtgtgcagaccgatttacgacatgaaggacgctcaacgaagagtttcgagggaactccaagcgagagggctggtggaaaacctttcccgcgaacaacttgttgaagctcaggcgcgcgtttcggtgtgccctcacactctcggcaattgtaacgtgaaaagcgacgtgctaatcatggtgcaggtttacgacgcttctttgaacgaaattgcgtcggcgatggtcctaaaggaatcgaaagtcgcttacctcaccatggtgactccgggtgaacttctagatgaacgtgaagccttcgctatcgacgctttggggtgcgacgtcgtcgtggacacccgccgagacatggtgcagtacaaattcggctcatcttgttactgccacaaactgtcgaatataaagaatataatgttgactcccgcattcactttcagcgggaatcttttttcggtcgagatgtacgaaaaccgaatgggcgtcaactactacaaaatcactcgttcagcgtattctcctgaaatacgcggcgttaagacgctgcggtaccgccgagcctgcaccgaggtcgtgcaagttaaactgccacgcttcgataaaactctaaagacgtttctctccgggtatgattacatatatttggatgcgaaattcgtctctcgagtcttcgattacgtggttagcaactgtagcgttgttaacagtaaaaccttcgagtgggtgtggagttacataaaatcaagtaaatcgcgcgttgtcataagtgggaaagtgatacatcgagatgtgcatatcgacctcaaacactctgaatgttttgcggctgtgatgctcgcggttggggtgcgttctcgcacaactactgaatttttagcgaagaacctcaattattacactggcgatgcttcgtgctttgagactatccgtttcctctttcgggagtggagccggagggcttacgcggaaataaatcgcagttttcgaaagcttatgaagagcattctttccgctgggttagattacgaatttctcgatcttgacaactcgctccaacacttgcttgaatactcagaggttgaagtgcgcgtttcaatcgctcagaatggtgaggtggattgcaacgaagagaaccgtgttttgacggagataatagccgaagcagcggacaggaaatccatcgctcaaggtctcagcggagcgttgagctccgttccaacgcaaccgagaggcggtctgagagggggtagtcgccgaagtggagtttttttcctttataacttggtagaggaagtcggaaatcttttcttttccgtcggtgatgccgtgcggtttcttgttaagttatttaaaactttttccgactctcccatctttcgggtcgttcggatgttcctggacttggcggaagcagcttcaccgttcgtttcggttgtttctttgtgcgcgtggttgcgcgaagctgtaagcgctttctcaggctgggttgccgacagaacggtgtcggaaagtgtaaaaacctttgtaaaccgtacggtaaaaaggtttctaaactttatgtctgcaaaaaccctgacgaaaaaattttttaggtttttcttatccgcttccgccttagctaaaaccgttgtgaggaaggcgaaggtgattttggaagcttactgggaagtgtggttcgaatcgattctttcggatagtggagagtatagtgctgttgaattttgtagcagcgttgtgatcacactgttaacgaattccggtcggctgctacctgggtttagtccatccgctgtcattactgaagtactgcttgacctagccacgaaaatatccattgaagttcttctaaaacaaatttctcctgtcgactcgacagcttcttcggccttatatcgccgcgtcctaagcgagattctgtcgaattttcgcacaatgggcgaacacgggatcttcactaaagtctttttactctgcgggtttctacccgtgtttgttaggaagtgcgtcgctctgtgcgttcctggtgacatggcgacgtacgcccggtttctcgaatacggggtcgacgatctcttctttttgggaaggtccgtgaactcgattaagaactacctatgtgtggttgctgccgggttggtggactccattgtcgattccgttgttttgaaactttccggtgtcgccaaagagcgagtgcttggttttaaatcaaaaatcataaaaaattttttaaacgtctttaggaaggcgaaagtcgttacacgtacttcttccagcacggacttgtcggaagacgaatatttttcgtgcgacgaaagcaaacccggcttgagaggcggttcctctaggttcacgctttcgcgattgcttgacatctttttcaatttcctgaaaagttcaaagctcgtcatagagaatgcgtgcttttctgcttacgaaaggattgaaaggaacatgaaactatacttttttcctttaaattcttcggaagaagaagctcgccgcttaattcggtgcgcgggagacttcgactacttaagcgatagcgctttcgacgaagatgaaatgttacggcaagctttcgaacaatactattccagtgacgacgagagtgtaacttacgatggaaaacccacagttctccgtagttacttgaacgtgagtagaaggttcttggaaacgttctgtaacggcccgaagtttttcgtcaaagtttcgaattactttaaagcgctgtatagtcgtttgctccgtgttctaccgtgggtagacagaaatctttctgactcgccaggacttaaaggaggtaacgagaaagctcttcttgcgaagttccttaaaacctgcgtcataaccgcttgcgaatgtgtgtcgcagatatgctgcctgcgtcttattcgcttatgttgggggacaccagcgtgtggtttagttaggttattttacataacttattccggtactcgcgtgttgtcgcgtgttgtggtcgcggtggctgtatgccctcttttggtcagaaacgaactggatggcttgagtgatggattaaccaacatgggcgtttcggtttttcgtcgcttgttcgtcgcccttcggcgcgcgctttcggcgtactctaattccgccctacgacggaagattttcgaattcattttcggaaacattcaccacccttttgatgtcgcagtaatcgaaacaaacgaagttgcgccggaacctctttcaccggaagtggacattgatgtcgactgcgactttggttccgattcagaatcggtttcttcagatgaagtcgcgtcgattccccgtccaggcttacacggtgggaataggcgctcttccaactttctaacctctctcgtgaaggttgtttttaagttggctgggcgcattccgcgtttgcttttccgtttacgcaattttgtggcgtactttgttgaacgacggctggcttcaaaaaggttgaagacgttcatcggtctggccaggctgtttgataatttctcactcacttcggtagtttaccttctccaagagtacgattcagtgctgaacgcatttatagacgttgagctagtcctacttaactcgggtagcgtgaatgtgctacctttagtttcttgggtcaggggatctctaacgaagttagcggaagtgatcgttggttcaggcttcgcttcgtttctaggaaggatgtgctgtcgtgtgtccgactggtgctcctcgtcttcgaacgccggttgtaactttatgagtccggttcgtacgaaagggaagttcgttcccccttcgtcttccggttcaaccgcttcgatgtatgaacgtctcgaagctctcgagagcgatatccgcgaacacgtgctttccacgtgtcgtgtaggaagcgacgaagaagaggaaaggccgaaagaagtgacagaaccaggaattgaacatacttctgaagatgttgttcccattcgttcacactcgcaacccttatctggaggtgaatgttcgtattctgaagatcgtgaagagaatgaacgagcgaacctgttaccgcacgttagcaaaatcgtcagcgaacgaaggggtttggagaccgcccgtcgaaacaaacgtactctacgtggtgtaagcgagtttctcaacgctattaatactagcaatgagcaacctaggccgataatcgttgaccactctcctgaatctcgcgcgttgaccaactccgtgagggaattctattacctccaggaactcgctctttttgagttgagttgcaaacttcgtgagtattacgatcaattgaaggttgcgaattttaacagacaagagtgtttgtgcgacaaagacgaagacatgtttgttctacgagccggacaaggtgtagtttccggcagaaactcgaggttgcctcttaagcatttcaaggatcacgaattttgttttcgctctggagggttggtcccttacgacggtaccagcagagtggacaccatttttcacacgcaaacgaatttcgtttccgcgaacgcgcttctttcgggctatctctcctatagaactttcactttcactaatttgagcgctaacgtactgctgtacgaagctcctccaggtggtggaaagaccacgactttgataaaggttttttgtgaaactttttcaaaagttaattcgctgattttaacggcgaataagagttcgcgagaagaaatacttgcgaaggtgaatcgcatcgtacttgacgaaggcgatacgcctcttcagacgcgtgacaggattttaactatcgattcttatctaatgaacaacagaggtttgacgtgtaaggttttgtacctcgatgagtgtttcatggttcacgctggagctgctgtagcttgcatcgaattcaccaaatgcgattcagccatcttatttggagacagcaggcaaattcactacatagaccgtaacgaattggatactgctgttctttcagatttgaaccgttttgtcgatgatgaatcgagagtatatggtgaagtctcatacaggtgtccttgggacgtttgtgcttggttgtcaactttctacccgaaaactgtggccactaccaacttggtttcagccggtcaatcttcgatgcaagtacgcgagattgaaagcgtagacgacgtcgaatattccagtgaattcgtctacttgactatgttacagtcagagaagaaagatctgctgaaatctttcggcaagaggtctcgttcgagcgttgaaaaacccacggtcttaacagtccatgaagctcaaggtgaaacctaccgcaaagttaacctcgtcagaacgaaatttcaagaggacgatccttttcgtagcgagaaccacatcacagtggccttgtctaggcatgtcgagagtctgacctattcggtcctgagtagtaaacgtgacgacgcaatagctcaagctatagcgaaggcgaaacaacttgtggatgcctatcgcgtttaccccacgtcatttggtgggagtactcttgatattagtgttaacccttctacatctgacaggagcaaatgtaaagcttcctctgccccttacgaagttataaacagcttcttggagagcgtggttccgggcactacttcagtagactttggggacgtttccgaagagatgggcactcaggtttttgagtccggtgctgataacgttgttattcgtgattccgcacctgttaacaagtcgacggatcacgacccgcagcgggtttagctcgattcgctcgcaggcgattcctaagaggaaaccgtcgctgcaagagaatttatactcttatgagtcgcgtaattacaactttaccgtttgtgaacgtttttccggaccgcaggagttcggacaggcgatggcgatggttatgttggaacgaagctttgacttagagaaagttgctaaagttagaagcgatgtgatcgccataacagaaaaaggggtgcgaacatggatgtcaaaacgtgaaccttctcagcttagggctcttagtagtgacttacaaaagcctctaaacttggaagaggaaataacgacttttaagttgatggttaagcgggacgcgaaagtcaaactcgattcgtcgtgtttggtgaaacacccaccagcgcagaatataatgttccatcgcaaggcggtgaacgcgattttctcgccgtgtttcgacgagtttaaaaatagagtcattacctgtacgaattcaaatattgttttctttaccgaaatgactaactctactctcgcgtcgatagcgaaagagatgctggggagcgaacacgtttacaacgttggggaaatagacttttcgaaattcgacaaatcccaagacgctttcattaagtcgtttgaacgaaccttgtattcagcgtttggtttcgacgaagacctgcttgacgtgtggatgcaaggtgaatacaccagcaacgctacaactctggacggtcaactttctttttccgtcgacaaccagaggaaatcgggcgcttctaatacgtggattggtaattccatcgtgactcttggcattttgagcatgttctattacaccaatcgatttaaggctcttttcgtgtccggggacgactctttgattttctccgaatctcctattagaaattcagccgatgcgatgtgcacagaactcggttttgagactaagtttctcactccgagcgtcccgtatttctgctcaaagtttttcgttatgaccggtcacgacgttttcttcgtgcccgacccttataaacttttagtgaaattaggagcttctaaggatgaagtggacgatgagtttctgtttgaagtgttcacctcttttcgcgatttaacgaaagatttagtcgatgaaagagtgatcgaactcttgacgcatttggttcacagtaagtacgggtacgaaagtggtgatacgtacgccgccctgtgtgctattcattgtattcgttcaaacttttcatcgttcaagaaattgtaccctaaagttaagggctgggtcgttcactacggtaaactgaagtttgtgctgcgcaaattcgcgaactgttttcgcgagaagtttgacactgctttcggcgaagcgtactttcttacttacgacgaaacttgagactgtgtggtaactcggttgttgttttgtttgtttgtgtgtctgtagtcctacttcgcggtgatgggatccttaattaaggcgcgcctgtacagctagctatggccggcctctagagaagtgacggaggtgacctacctgccgtgtaatttaagtcgtcacagagtgacaacggcaccaagtggtgctttgtgcgtatgtaaattgcgaagtgaaaaaattttttaaaaaaatttttcacactcctagcgaagtcccgctaggaagaaaaaataagggccgtcaagacatgattcacatgtcttctgatgagtccgtgaggacgaaacggcccggggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacagaatatagcgcgcaaactaggataaattatcgcgcgcggtgtcatctatgttactagatcaagcttatcgataccgtcgacctcgagggggggcccggtaccaaaaccaccccagtacattaaaaacgtccgcaatgtgttattaagttgtctaagcgtcaatttgtttacaccacaatatatcctgcca

SEQ ID NO: 3 - DNA, 236 bp, Beet yellows virus
cgtcagggtatgagttcctcggggcagattttctaactgcgaccagcgtgtgtttgaacgatcacgaaaaagctatcgtactacaggcctcaagagctgccattgatagagcagtctcttcgtcggtcgacgggaagatcgtcagtcttttcgacctcggtcgtcttagttaacacagttactaaggttccattttattattgcattgtttttcatttagtgtaatcgtacttgag

SEQ ID NO: 4 - DNA, 392 bp, Beet yellow stunt virus
aaccaaatggtttaagggctttttgcgcctctttagaaggaatgtacctttctgtagctcgactgggcccggacgcgttcggcactaggtccgttgggaagcgtggtgcgccttcaggaagcgagtatttaggcgccgattttctgacctcaacgtgtccgttaatgtccgaccacgatcgtgctgtagctttgagtgcatctagaaacgcactcgaccgatctgcggcgtctcaaattgataaaaagatggttagtttgtacgacttcggtaaggtagtgtatacctagcagcttgcttctacctgacacagttaagaagcggcataaatcgaagccaaaccctaaattttgcaactcgatcaattgtaacctagagcgaagtgcaatcat

SEQ ID NO: 5 - DNA, 378 bp, grapevine leafroll-associated virus 2
gatgaaaagtctcacgaagaagtacaaacgagtgaatggtctgcgtgcgttctgttgcgcgtgcgaagatctatatctaaccgtcgcaccaataatgtcagaacgctttaagactaaagccgtagggatgaaaggtttgcctgttggaaaggaatacttaggcgccgactttctttcgggaactagcaaactgatgagcgatcacgacagggcggtctccatcgttgcagcgaaaaacgctgtcgatcgtagcgctttcacgggtggggagagaaagatagttagtttgtatgatctagggaggtactaagcacggtgtgctatagtgcgtgctataataataaacactagtgcttaagtcgcgcagaagaaaacgct

SEQ ID NO: 6 - protein, 245 aa, Artificial Sequence
  Synthetic
MGFVLFSQLPSFLLVSTLLLFLVISHSCRAEIVLTQSPGTLSLSPGERATLSCRASQSVSYSSLAWYQQKPGQAPSLLIYGASSRATGIPDRFSGSGSGPDFTLTISRLEPEDFAVYYCQHYGNSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 738 bp, Artificial Sequence
  synthetic
atgggatttgttctcttttcacaattgccttcatttcttcttgtctctacacttctcttattcctagtaatatcccactcttgccgtgccgaaattgtgcttactcagtctccaggaactctttctctttctccaggtgaaagggctactctttcttgcagggcttctcagtctgtgtcttactcttctcttgcttggtatcagcaaaagccaggacaagctccatctcttcttatttacggtgcttcttctagggctactggtattccagataggttctctggatctggatctggtccagatttcactcttactatttctaggcttgagccagaggatttcgctgtttactactgccagcactacggaaattctccatacactttcggacagggaactaagcttgagattaagaggactgtggctgctccatctgtgtttattttcccaccatctgatgagcaacttaagtctggaactgcttctgttgtgtgccttcttaacaacttctacccaagggaagctaaggttcagtggaaagtggataacgctcttcagtctggaaactctcaagagtctgtgactgagcaggattctaaggattcaacttactctctttcttctactcttactttgtctaaggctgattacgagaagcacaaggtttacgcttgcgaagttactcatcagggactttcttctccagtgactaagtctttcaacaggggagagtgctga

SEQ ID NO: 8 - protein, 479 aa, Artificial Sequence
  Synthetic
MGFVLFSQLPSFLLVSTLLLFLVISHSCRAQVQLVQSGAEVKKPGASVKVSCKASGYTFTSNAIQWVRQAPGQRLEWVGWINGGDGNTKYSQKFQGRVTISRDISASTAYMELSSLRSEDTAVYYCARHRLQRGGFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1440 bp, Artificial Sequence
  Synthetic
atgggatttgttctcttttcacaattgccttcatttcttcttgtctctacacttctcttattcctagtaatatcccactcttgccgtgcccaggttcagcttgttcaatctggtgctgaggttaagaaacctggtgcttctgttaaggtgtcatgcaaggcttcaggatacactttcacttctaacgctattcagtgggttaggcaagctccaggacaaagacttgaatgggttggctggattaacggtggagatggaaacactaagtactctcagaagttccagggaagggttacaatttctagggatatttctgcttctactgcttacatggaactttcttctcttagatctgaggatactgctgtgtattactgcgctaggcatagacttcaaaggggaggatttgatccttggggacagggaactcttgtgactgtgtcatctgcttcaactaagggaccatctgtgtttccacttgctccatcttctaagtctacttcaggtggaactgctgctcttggatgccttgtgaaggattacttcccagagccagttactgtttcttggaactctggtgctcttacttctggtgttcacactttcccagctgtgcttcagtcatctggactttactcactttcttcagtggtgactgtgccttcttcttctcttggaactcagacttacatctgcaacgtgaaccacaagccatctaacacaaaagtggataagagggtggagccaaagtcttgcgataagactcatacttgtccaccatgtccagctccagaacttcttggaggaccttctgtgttccttttcccaccaaagccaaaggatactcttatgatttctaggactccagaggttacatgcgttgtggttgatgtgtctcatgaggatccagaggtgaagttcaactggtacgtggatggtgttgaggttcacaacgctaagactaagccaagggaagagcagtacaactctacttacagggttgtgtctgtgcttactgtgcttcaccaggattggcttaacggaaaggaatacaagtgcaaggtgtcaaacaaggctcttccagctccaattgaaaagactatttctaaggctaagggacaacctagagagccacaggtttacactcttccaccatctagggaagagatgactaagaaccaggtgtcacttacttgcttggtgaagggattctacccatctgatattgctgttgagtgggagtctaatggacaaccagagaacaactacaagactactccaccagtgcttgattctgatggatctttcttcctttactctaagcttactgtggataagtctaggtggcagcagggaaatgttttctcttgctctgtgatgcatgaggctcttcacaatcactacactcagaagtctttgtctctttctcctggaaagtga

SEQ ID NO: 10 - protein, 777 aa, Artificial Sequence
  Synthetic
MGFVLFSQLPSFLLVSTLLLFLVISHSCRAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIGKLHHHHHHKDEL

SEQ ID NO: 11 - DNA, 2334 bp, Artificial Sequence
  Synthetic
atgggatttgttctcttttcacaattgccttcatttcttcttgtctctacacttctcttattcctagtaatatcccactcttgccgtgccgaggtgaagcaagagaataggcttcttaatgagtctgagtcatcttctcagggattgcttggttactacttctctgatcttaatttccaggctcctatggtggttacttcttctactactggtgatctttctattccttcttctgagcttgagaatattccttctgagaatcagtacttccagtctgctatttggagtggtttcattaaggtgaagaaatctgatgagtacactttcgctacttcagctgataatcatgtgactatgtgggtggacgatcaggaggtgatcaataaggcttctaattctaataagattaggcttgagaagggaaggctttaccagattaagattcagtaccagagggaaaatcctactgagaagggtcttgatttcaagttgtactggactgattcacagaataagaaagaagtgatttcttctgataatcttcagcttcctgagcttaagcagaagtcatctaattctaggaagaagaggtctacttctgctggtcctactgttcctgatagggataatgatggtattcctgattctcttgaggtggagggttacactgtggatgtgaagaataagaggactttcctttctccttggatttctaatattcatgagaagaagggtcttactaagtacaagtcatctcctgagaagtggtctactgcttctgatccttactctgatttcgagaaggtgacaggaaggattgataagaatgtgtctcctgaggctagacatcctcttgttgctgcttaccctattgtgcatgtggatatggagaatattattctttctaagaatgaggatcagtctactcagaatactgattctgagactaggactatttctaagaatacttctacttctaggactcatacttctgaagtgcatggaaatgctgaagttcatgcttctttcttcgatattggtggttctgtgtctgctggtttctctaattcaaattcttctactgtggctattgatcattctctttctcttgctggtgaaaggacttgggctgagactatgggacttaatactgcagatactgctaggcttaatgctaatattagatacgtgaatactggtactgctcctatctacaatgtgcttcctactacttctttggtgcttggaaagaatcagactcttgctactattaaggctaaagagaatcagctttctcagattcttgctcctaacaattactacccttctaagaatcttgctccaattgctcttaatgctcaggatgatttctcttctactcctattactatgaattacaatcagttccttgagcttgaaaagactaagcagcttaggcttgatactgatcaggtgtacggtaatattgctacttacaatttcgagaatggtagagtgagagtggatactggttctaattggagtgaggtgttgcctcagattcaagagactactgctaggattattttcaatggaaaggatcttaatcttgtggagagaaggattgctgctgtgaatccttctgatcctcttgagactactaagcctgatatgactcttaaagaggctcttaagattgctttcggtttcaatgagcctaatggaaatcttcagtaccagggaaaggatattactgagttcgatttcaatttcgatcagcagacttctcagaacattaagaaccagctagctgagttgaatgctactaatatctacactgtgttggataagattaagttgaatgcaaagatgaatattcttattagggataagaggttccattacgataggaacaatattgctgtgggtgctgatgagtctgttgtgaaagaggctcatagggaagttatcaattcttcaactgagggacttcttcttaatattgataaggatattaggaagattctttctggttacattgtggagattgaggatactgagggtcttaaagaagtgatcaatgatagatacgatatgttgaatatttcttctcttaggcaggatggaaagactttcattgatttcaaaaagtacaatgataagttgcctctttacatttctaatcctaattacaaagtgaatgtgtacgctgtgacaaaagagaacactattatcaatccatctgagaatggtgatacttctactaatggtattaagaagattttgattttctctaagaagggttacgagattggaaagcttcaccaccatcatcatcataaggatgaactttga

SEQ ID NO: 12 - protein, 441 aa, Artificial Sequence
  Synthetic
MGFVLFSQLPSFLLVSTLLLFLVISHSCRANNDFCKPSSLNSEISGFIGYKCNFSNEGVHNLKPDMRERRSIFCTIHSYFIYDKIRLIIPKKSSSPEFKILPEKCFQKVYTDYENRVETDISELGLIEYEIEENDTNPNYNERTITISPFSPKDIEFFCFCDNTEKVISSIEGRSAMVHVRVLKYPHNILFTNLTNDLFTYLPKTYNESNFVSNVLEVELNDGELFVLACELINKKCFQEGKEKALYKSNKIIYHKNLTIFKAPFYVTSKDVNTECTCKFKNNNYKIVLKPKYEKKVIHGCNFSSNVSSKHTFTDSLDISLVDDSAHISCNVHLSEPKYNHLVGLNCPGDIIPDCFFQVYQPESEELEPSNIVYLDSQINIGDIEYYEDAEGDDKIKLFGIVGSIPKTTSFTCICKKDKKSAYMTVTIDSAHHHHHHKDEL

SEQ ID NO: 13 - DNA, 1326 bp, Artificial Sequence
  Synthetic
atgggattcgtgcttttctctcagcttccttctttccttcttgtgtctactcttcttcttttccttgtgatttctcactcttgtagggctaacaacgatttctgcaagccatcttctcttaactctgagatttctggattcattggatacaagtgcaacttctctaacgagggtgttcacaaccttaagccagatatgagagagagaagatcaattttctgcactattcactcttacttcatttacgataagattaggcttattattccaaagaagtcatcttctccagagttcaagattcttccagagaagtgcttccagaaggtgtacactgattacgagaacagggtggagactgatatttctgagcttggacttattgagtacgagattgaagagaacgatacaaacccaaactacaacgagaggactattactatttctccattctctccaaaggatattgagttcttctgcttctgcgataacactgagaaagtgatttcttctattgagggaagatcagctatggttcatgtgagggtgttgaagtacccacacaacattcttttcactaaccttactaacgatcttttcacttacttgccaaagacttacaacgagtctaacttcgtgtctaacgtgcttgaggtggagcttaatgatggtgagttgttcgttcttgcttgcgagcttattaacaagaagtgtttccaagagggaaaagagaaggctctttacaagtctaacaagattatttaccacaagaaccttactattttcaaggctccattctacgtgacttctaaggatgtgaacactgagtgcacttgcaagttcaagaacaacaactacaagattgtgcttaagccaaagtacgagaagaaagtgattcacggatgcaacttctcatctaacgtgtcatctaagcacactttcactgattctcttgatatttctcttgtggatgattctgctcacatttcttgcaacgtgcacctttctgagccaaagtacaaccaccttgtgggacttaattgcccaggtgatattattccagattgcttcttccaggtttaccaaccagagtctgaagaacttgagccatctaacattgtgtaccttgattctcagattaacattggagatattgagtactacgaggatgctgagggtgatgataagattaagttgttcggaattgtgggatctattccaaagactacttctttcacttgcatctgcaagaaggataagaaatctgcttacatgactgtgactattgattcagctcatcaccatcaccaccacaaggatgagctttga

SEQ ID NO: 14 - protein, 355 aa, Artificial Sequence
  Synthetic
MGFVLFSQLPSFLLVSTLLLFLVISHSCRAAPADNTVNIKTFDKVKNAFGDGLSQSAEGTFTFPADVTTVKTIKMFIKNECPNKTCDEWDRYANVYVKNKTTGEWYEIGRFITPYWVGTEKLPRGLEIDVTDFKSLLSGNTELKIYTETCLAKGREYSVDFDIVYGTPDYKYSAVVPVIQYNKSSIDGVPYGKAHTLGLKKNIQLPTNTEKAYLRTTISGWGHAKPYDAGSRGCAEWCFRTHTIAINNANTFQHQLGALGCSANPINNQSPGNWAPDRAGWCPGMAVPTRIDVLNNSLTGSTFSYEYKFQSWTNNGTNGDAFYAISSFVIAKSNTPISAPVVTNDYKDDDDKDEL

SEQ ID NO: 15 - DNA, 1068 bp, Artificial Sequence
  Synthetic
atgggtttcgtgctgttctctcagcttccatctttccttttggtgtctacccttcttctgttccttgtgatttctcattcttgcagagctgctccagctgataacaccgtgaacattaagaccttcgataaggtgaagaacgctttcggtgatggtctttctcaatctgctgagggaacttttaccttccctgctgatgtgactaccgttaagaccatcaagatgttcatcaagaacgagtgccctaacaagacttgtgatgagtgggataggtacgctaatgtgtacgtgaagaacaagactactggtgagtggtatgagatcggtagattcattactccttactgggtgggaactgagaagcttcctagaggtcttgagattgatgtgaccgatttcaagtctctgctgtctggtaataccgagcttaagatctacaccgagacttgtcttgctaagggtagagagtactccgttgatttcgatattgtgtacggaacccctgattacaagtactcagctgttgttcctgtgatccagtacaacaagtctagcattgatggtgtgccatacggtaaggctcatactcttggtctgaagaagaacattcagttgcctactaacaccgagaaggcttatcttaggaccactatttctggttggggtcatgctaagccttatgatgctggttctagaggttgtgctgagtggtgttttaggactcataccattgctatcaacaacgctaacactttccagcatcagcttggtgctcttggttgttctgctaaccctattaacaaccagtctcctggtaattgggctcctgatagagctggttggtgtcctggtatggctgttcctactaggattgatgtgctgaacaactctcttaccggttctacattcagctacgagtacaagttccagtcttggactaacaacggtactaacggtgatgctttctacgctattagctctttcgtgatcgctaagtctaatacccctatttctgctcctgtggtgaccaatgattacaaggatgatgatgataaggatgagctttag

(19) United States
(12) Patent Application Publication
(10) Pub. No.: US 2014/0056908 A1
(43) Pub. Date: Feb. 27, 2014

(54) CLOSTEROVIRUS-BASED NUCLEIC ACID MOLECULES AND USES THEREOF

(71) Applicants: Alexei Prokhnevsky, Wilmington, DE (US); Vidadi Yusibov, Havertown, PA (US); Vadim Mett, Newark, DE (US)

(72) Inventors: Alexei Prokhnevsky, Wilmington, DE (US); Vidadi Yusibov, Havertown, PA (US); Vadim Mett, Newark, DE (US)

(73) Assignee: FRAUNHOFER USA INC., Newark, DE (US)

(21) Appl. No.: 13/878,104

(22) PCT Filed: Oct. 7, 2011
(86) PCT No.: PCT/US11/55365

§ 371 Date: Nov. 7, 2013

Related U.S. Application Data
(60) Provisional application No. 61/391,333, filed on Oct. 8, 2010.

(51) Int. Cl. C07K 16/12; C07K 14/32

(52) U.S. Cl. CPC C07K 16/1278; C07K 14/32
USPC 424/150.1; 424/246.1; 424/94.6; 435/69.1; 435/227; 435/320.1; 514/1.1

(57) ABSTRACT

The present invention relates to novel nucleic acid molecules for producing target polypeptides in plant cells. More specifically, the novel nucleic acid molecules comprise a minireplicon derived from a Closteroviridae virus and heterologous polynucleotides encoding the target polypeptides. Also provided are compositions comprising the target polypeptides and uses thereof.

### CROSS-REFERENCE TO RELATED APPLICATION

This application claims the benefit of U.S. Provisional Application No. 61/391,333, filed Oct. 8, 2010, the contents of which are incorporated herein by reference in their entireties for all purposes.

### FIELD OF THE INVENTION

The present invention relates generally to novel nucleic acid molecules for producing target polypeptides in plant cells. More specifically, the nucleic acid molecules comprise a minireplicon derived from a Closteroviridae virus and polynucleotides encoding the target polypeptides.

### BACKGROUND OF THE INVENTION

Production of recombinant proteins, including monoclonal antibodies (mAbs), vaccine antigens, enzymes, dual vaccines, fusion molecules and virus-like particles (VLPs), using plant viral vectors is an attractive alternative to traditional mammalian cell-based expression systems. Due to the high speed of virus replication, plant viral vectors have the potential to rapidly produce large quantities of foreign proteins.

A tobacco mosaic virus (TMV)-based vector is the most widely used vector for transient expression of plant-produced subunit vaccines and therapeutic proteins (Streatfield; Yusibov at al.; Gleba at al.; Rybicki). However, expression of high molecular weight proteins or co-expression of multiple polypeptide chains or proteins using TMV vectors is challenging. There remains a need for improved plant viral vectors for producing single large target proteins as well as simultaneously producing multiple target proteins in plants.

### SUMMARY OF THE INVENTION

The disclosed subject matter of the present invention relates to novel nucleic acid molecules for producing target polypeptides in plant cells.

According to one aspect of the present invention, an isolated nucleic acid molecule for producing one or more target polypeptides in a plant cell is provided. The nucleic acid comprises a minireplicon derived from a Closteroviridae virus and one or more heterologous polynucleotides. The nucleic acid molecule is capable of replicating in the plant cell. The one or more heterologous polynucleotides encode the one or more target polypeptides. The nucleic acid molecule may further comprise a polynucleotide encoding one or more movement proteins derived from the Closteroviridae virus. The Closteroviridae virus may be Beet yellows virus.

The plant cell may be in a plant, a plant part, or a cell culture medium. The plant may be a whole growing plant. The plant part may be selected from the group consisting of leaves, stems, roots, floral tissues, seeds and petioles.

In one embodiment, the one or more target polypeptides comprise one or more subunits of a protein, and are capable of forming the protein in the plant cell. The protein may be an enzyme.

In another embodiment, the one or more target polypeptides comprise a first polypeptide and a second polypeptide, and the first polypeptide is capable of modifying the second polypeptide in the plant cell.

In yet another embodiment, the one or more target polypeptides comprise a first polypeptide and a second polypeptide, and the first polypeptide is capable of affecting expression of the second polypeptide in the plant cell. The first polypeptide may be a silencing suppressor.

In a further embodiment, the one or more target polypeptides comprise a first polypeptide and a second polypeptide, and the first polypeptide is capable of increasing production of the second polypeptide in the plant cell.

In yet a further embodiment, the one or more target polypeptides comprise a heavy chain and a light chain of an antibody, and are capable of forming the antibody in the plant cell.

The one or more target polypeptides may comprise an immunogenic polypeptide. The one or more target polypeptides comprise a polypeptide of at least 100 kD.

For each nucleic acid molecule of the present invention, a vector comprising the nucleic acid molecule is provided.

According to another aspect of the present invention, a method for producing one or more target polypeptides in a plant cell is provided. The method comprises (a) introducing a nucleic acid molecule into the plant cell; and (b) maintaining the plant cell under conditions permitting production of the one or more target polypeptides in the plant cell. The nucleic acid molecule comprises a minireplicon derived from a Closteroviridae virus and one or more heterologous polynucleotides. The nucleic acid molecule is capable of replicating in the plant cell. The one or more heterologous polynucleotides encode the one or more target polypeptides. The one or more target polypeptides comprise a polypeptide of at least 100 kD. The method may further comprise purifying at least one of the one or more target polypeptides from the plant cell. The nucleic acid molecule may further comprise a polynucleotide encoding one or more movement proteins derived from the Closteroviridae virus. The Closteroviridae virus may be Beet yellows virus.

In the production method of the present invention, the plant cell may be in a plant, a plant part, or a cell culture medium. The plant may be a whole growing plant. The plant part may be selected from the group consisting of leaves, stems, roots, floral tissues, seeds and petioles.

A composition comprising at least one of the one or more target polypeptides produced by the method of the present invention is provided. A method of treating a subject in need of at least one of the one or more target polypeptides produced thereby is also provided. The treatment method comprises administering to the subject an effective amount of a pharmaceutical composition comprising the at least one of the one or more target polypeptides.

In one embodiment, the one or more target polypeptides comprise one or more subunits of a protein. In the production method, the maintaining conditions further permit production of the protein in the plant cell. A composition comprising the protein produced thereby is provided. A method of treating a subject in need of the protein is also provided. The treatment method comprises administering to the subject an effective amount of a pharmaceutical composition comprising the protein. The protein may be an enzyme.

In another embodiment, the one or more target polypeptides comprise a first polypeptide and a second polypeptide. In the production method, the maintaining conditions further permit modifying, affecting expression, and/or increasing production of the second polypeptide by the first polypeptide in the plant cell. A composition comprising the second polypeptide is provided. A method of treating a subject in need of the second polypeptide is also provided. The treatment method comprises administering to the subject an effective amount of a pharmaceutical composition comprising the second polypeptide. The first polypeptide may be a silencing suppressor.

In yet another embodiment, the one or more target polypeptides comprise a heavy chain and a light chain of an antibody. In the production method, the maintaining conditions further permit production of the antibody in the plant cell. A composition comprising the antibody is provided. A method of treating a subject in need of the antibody is also provided. The treatment method comprises administering to the subject an effective amount of a pharmaceutical composition comprising the antibody.

In a further embodiment, the one or more target polypeptides comprise an immunogenic polypeptide. A composition comprising the immunogenic polypeptide produced thereby is provided. A method for inducing an immune response in a subject is also provided. The induction method comprises administering to the subject an effective amount of a pharmaceutical composition comprising the immunogenic polypeptide. A method for inducing a protective immune response against a pathogen in a subject is further provided. The protective induction method comprises administering to the subject an effective amount of a pharmaceutical composition comprising the immunogenic polypeptide. The immunogenic polypeptide is derived from the pathogen.

### BRIEF DESCRIPTION OF THE DRAWINGS

FIG. 1 is a diagram illustrating the organization of the BYV genome. L-Pro-papain-like leader proteinase; Met, Hel, and Pol-methyltransferase, RNA helicase, and RNA-dependent RNA polymerase domains of the replicase, respectively; p6-6 kD protein; Hsp70h-a Hsp70 homolog; p64-64 kD protein; CPm and CP-minor and major capsid proteins, respectively; p20 and p21-20 and 21 kD proteins, respectively.

FIG. 2 is a diagram illustrating a T-DNA region of a BYV-based launch vector encoding Hc and Lc of an anti-PA antibody. 35S—Cauliflower mosaic virus 35S promoter; NOS—Nopaline synthase terminator; LB and RB—left and right borders of T-DNA, respectively.

FIG. 3A shows N. benthamiana leaves systematically infected with the BYV-based launch vector encoding Hc and Lc of an anti-PA antibody. FIG. 3B shows Western blot analysis of Hc, Lc and total anti-PA IgG expression in the systematically infected N. benthamiana leaves at 30, 32, 34, 36 and 39 days post infiltration (dpi). The maximum expression of total anti-PA IgG was observed on day 34, with 53 mg/kg of fresh leaf weight. *Standards, ng of total human IgG.

FIG. 4 is a diagram illustrating a T-DNA region of a T-DNA-based BYV minireplicon vector for expression of Hc and Lc of an anti-PA antibody. The Hc and Lc genes are under the control of the BYV CP promoter and the GLRaV2 CP promoter, respectively.

FIG. 5 is a diagram illustrating a T-DNA region of a T-DNA-based BYV minireplicon vector for expression of Hc and Lc of the anti-PA antibody. The Hc and Lc genes are under the control of the BYV CP promoter and BYSV CP promoter, respectively.

FIG. 6A-B shows Western blot analysis of Hc and Lc expression in N. benthamiana leaves systematically infected by a modified miniBYV vector at 5 and 7 dpi, respectively. FIG. 6C shows calculated amounts of Lc and Hc expression in the systematically infected N. benthamiana leaves at 5, 7 and 9 dpi. Lanes: 100, 50 and 25 (ng) of the human mAb standard. Q3, #1, Q3, #2, Q3, #3, Q4, #1, Q4, #2, Q4, #3-different clones of the miniBYV replicon carrying Hc and Lc of the anti-PA mAb.

FIG. 7 is a diagram illustrating a T-DNA region of a miniBYV replicon vector for expression of the anthrax Protective Antigen 83 (PA83) under the control of the BYV promoter.

FIG. 8A shows Western blot analysis of PA83 expression in systematically infected N. benthamiana leaves at 5, 7 and 9 dpi. FIG. 8B shows the amounts of total protein (TP) and total soluble protein (TSP) expression in the systematically infected N. benthamiana leaves at 5, 7 and 9 dpi. Lanes 1-9: 1, PA standard, 50 ng; 2, PA standard, 25 ng; 3, PA standard, 10 ng; 4, pCB-miniBYV-PA83, TP; 5, pCB-miniBYV-PA83, TSP; 6, pGR-D4-PA83, TP*; 7, pGR-D4-PA83, TSP*; 8, pClean 238-PA83, TP; 9, pClean238-PA83, TSP. * Without a silencing suppressor P1HcPro.

FIG. 9 is a diagram illustrating a T-DNA region of a miniBYV replicon vector for co-expression of target protein Pfs48 and an enzyme capable of modifying the target protein.

FIG. 10 shows co-expression of target protein Pfs48 and an enzyme PNGaseF capable of deglycosylating target protein Pfs48. Detection was made using anti-His antibody (A) and anti-FLAG antibody (B), respectively.

FIG. 11 is a diagram illustrating a T-DNA region of a miniBYV replicon vector for expression of three open reading frames (ORFs) for three different targets using a combination of strong and weak closteroviral promoters (i.e., BYV CP promoter, GLRaV2 CP promoter, and BYSV CP promoter).

FIG. 12A-H shows the nucleic acid sequence of a T-DNA region of a BYV launch vector (SEQ ID NO: 1) according to some embodiments of the disclosed subject matter. A BYV sequence (upper case) with multiple cloning sites (bold) along with the 35S promoter (lower case) and the NOS terminator (lower case italic) are introduced between the left border (LB) and right border (RB) of the T-DNA sequence (underline). The multiple cloning sites (bold) include Pad (14,254-14,261 bp), AscI (14,262-14,269), BsrGI (14,270-14,275), NheI (14,276-14,281) and FseI (14,285-14,292).

FIG. 13A-E shows the nucleic acid sequence of a T-DNA region of a miniBYV launch vector (SEQ ID NO: 2) according to some embodiments of the disclosed subject matter. A miniBYV sequence (upper case) with multiple cloning sites (bold) along with the 35S promoter with a dual enhancer (lower case) and the NOS terminator (lower case italic) are introduced between the left border (LB) and right border (RB) of the T-DNA sequence (underline). The multiple cloning sites (bold) include BamHI (10,278-10,285), Pad (10,286-10,293), AscI (10,294-10,301), BsrGI (10,302-10,307), NheI (10,308-10,313) and FseI (10,317-10,324).

FIG. 14 shows the nucleic acid sequences of (A) BYV CP promoter (SEQ ID NO: 3), (B) BYSV CP promoter (SEQ ID NO: 4), and (C) GLRaV2 CP promoter (SEQ ID NO: 5).

FIG. 15 shows (A) an amino acid sequence of Lc of an anti-PA antibody with the PR1a signal peptide from Nicotiana tavacum on the N-terminus (underline) and (SEQ ID NO: 6), and (B) a corresponding nucleic acid sequence with a TGA stop codon (underline) (SEQ ID NO: 7).

FIG. 16 shows (A) an amino acid sequence of Hc of an anti-PA antibody with the PR1a signal peptide from Nicotiana tavacum on the N-terminus (underline) (SEQ ID NO: 8) and (B) a corresponding nucleic acid sequence with a TGA stop codon (underline) (SEQ ID NO: 9).

FIG. 17 shows (A) an amino acid sequence of PA83 with the PR1a signal peptide from Nicotiana tavacum on the N-terminus (underline) and 6H is (bold) for purification and KDEL (italic) as an ER retention signal on the C-terminus (SEQ ID NO: 10), and (B) a corresponding nucleic acid sequence with a TGA stop codon (underline) (SEQ ID NO: 11).

FIG. 18 shows (A) an amino acid sequence of Pfs48 with the PR1a signal peptide from Nicotiana tavacum on the N-terminus (underline) and 6H is (bold) for purification and KDEL (italic) as an ER retention signal on the C-terminus (SEQ ID NO: 12) and (B) a corresponding nucleic acid sequence with a TGA stop codon (underline) (SEQ ID NO: 13).

FIG. 19 shows (A) an amino acid sequence of PNGaseF with the PR1a signal peptide from Nicotiana tavacum on the N-terminus (underline) and a FLAG tag (bold) for detection and KDEL (italic) as an ER retention signal on the C-terminus (SEQ ID NO: 14) and (B) a corresponding nucleic acid sequence with a TGA stop codon (underline) (SEQ ID NO: 15).

### DETAILED DESCRIPTION OF THE INVENTION

The present invention is based on the discovery that novel nucleic acid molecules comprising a Beet yellows virus (BYV) minireplicon can be used to produce a single or multiple heterologous target polypeptides in a plant cell. The BYV minireplicon may be used for production of therapeutically active proteins, subunit vaccines, protein adjuvants, enzymes, monoclonal antibodies (mAbs) and virus-like particles (VLP).

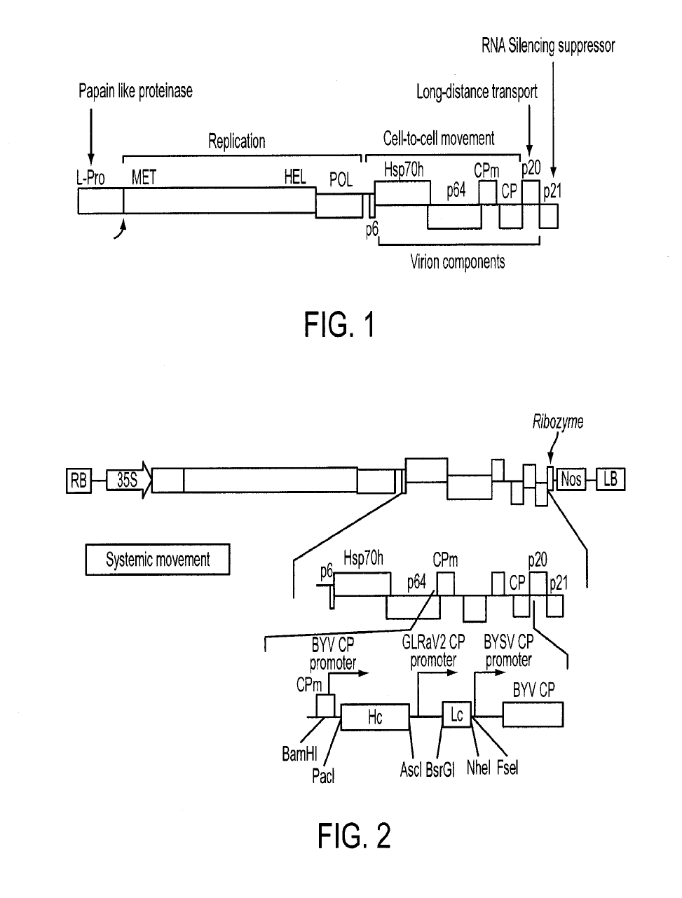

BYV is a member of the alphavirus supergroup of positive-strand RNA viruses belonging to the genus Closterovirus, family Closteroviridae. The 15.5 kb monopartite genome of BYV encodes 8 open reading frames (ORFs) (FIG. 1). Three groups of proteins are recognized in the BYV genome. The first group of proteins is responsible for virus replication, and includes methyltransferase (Met), helicase (Hel), and RNA polymerase (Pol) (ORF 1A and 1B). The second group of proteins is responsible for the virus cell-to-cell movement (ORFs 2-6), and includes P6, HSP70h, CP, CPm and p64. The knockout of any one of these proteins results in an arrest of the virus cell-to-cell movement. The third group of proteins includes viral structural components such as Hsp70H, CP, CPm, p64 and p20 (ORFs 3-7). p20 also known as the viral long distance transport factor. p21—the BW silencing suppressor (ORF 8).

BYV contains a replication gene block which covers more than 50% of the BYV genome and includes genes necessary for BYV replication. The BYV replication gene block is formed by the domain of papain-like leader proteinase (L-Pro), methyltransferase (Met), helicase-like domain region of viral replicase (Hel), and RNA-depended RNA polymerase (Pol). RNA-depended RNA polymerase is expressed from +1 frameshift. A larger replication protein which contains methyltransferase, helicase, and polymerase is produced in smaller quantities compared to the methyltransferase-helicase polyprotein due to the low frequency of frameshifting (FIG. 1). Flexious BYV virions are ˜1300 nm in length and ˜12 nm in diameter, and contain five structural proteins. The major capsid protein (CP) encapsidates ˜95% of the virion body. A short virion tail which is necessary for the BYV cell-to-cell and systemic movement contains minor CP (CPm); Hsp70h, a homolog of cellular heat shock proteins; p64, a 64 kD protein with unknown functions; and p20, a long distance transport factor. Other proteins of BW are p6, a small transmembrane protein required for BYV cell-to-cell movement and localized in the endoplasmic reticulum of host cell; and p21, a BYV silencing suppressor involved in binding of short interfering RNA.

The term “protein” used herein refers to a biological molecule comprising amino acid residues. A protein may comprise one or more polypeptides. Each polypeptide may be a subunit of protein. For example, the protein may be an antibody consisting of two Hc and two Lc. The protein may be in a native or modified form, and may exhibit a biological function when its polypeptide or polypeptides are properly folded or assembled.

The term “polypeptide” used herein refers to a polymer of amino acid residues with no limitation with respect to the minimum length of the polymer. Preferably, the polypeptide has at least 20 amino acids. A polypeptide may be a full-length protein, or a fragment or variant thereof.

The term “fragment” of a protein as used herein refers to a polypeptide having an amino acid sequence that is the same as a part, but not all, of the amino acid sequence of the protein. Preferably, a fragment of a protein retains the same function as the protein.

The term “variant” of a protein as used herein refers to a polypeptide having an amino acid that is the same as the amino acid sequence of the protein except having at least one modification, for example, glycosylation, phosphorylation, a deletion, an addition or a substitution. The variant may have an amino acid at least about 80%_{,} 90%, 95%, or 99%, preferably at least about 90%, more preferably at least about 95%, identical to the amino acid sequence of the protein. Preferably, a variant of a protein retains the same function as the protein.

The term “derived from” used herein refers to the origin or source, and may include naturally occurring, recombinant, unpurified or purified molecules.

According to one aspect of the present invention, a nucleic acid molecule for producing one or more target polypeptides in a plant cell is provided. The nucleic acid molecule comprises a minireplicon derived from a Closteroviridae virus and one or more heterologous polynucleotides, and is capable of replicating in the plant cell. The one or more heterologous polynucleotides encode the one or more target polypeptides.

A Closteroviridae virus may be any virus in the family of Closteroviridae. For example, the Closteroviridae virus may be Beet Yellows virus, Grapevine leafroll-associated virus 2 (GLRaV2), Beet yellows stunt virus (BYSV), Citrus tristeza virus (CTV), Carrot yellow leaf virus (CYLV), or Lettuce infectious yellows virus (LIYV). Preferably, the Closteroviridae virus is Beet yellows virus.

A plant cell may be a cell in any plants, plant parts (e.g., leaves, stems, roots, floral tissues, seeds and petioles) or cell culture media. The plant may be a whole growing plant. The cell culture media may be any media suitable for growing plant cells, preferably in suspension. The plant cell is preferably susceptible to infection by a Closteroviridae virus. More preferably, the plant cell is susceptible to BW infection. The plant cell is preferably suitable for expression of a target polypeptide. For example, the plant cell may be cells in N. benthamiana leaves. Other suitable plants include Nicotiana clevelandii, Beta vulgaris, Spinacia oleracea, Brassica spp, Lactuca sativa, Pisum sativum, Nicotiana tabacum, Plantago lanceolata, Tetragonia tetragonioides, Montia perfoliata, Beta vulgari, Spinacia oleracea, Stellaria media, Brassica spp., Lactuca sativa, Pisum sativum, Nicotiana tabacum, Plantago lanceolata, Montia perfoliata, Tetragonia tetragonioides, Chenopodium foliosum, and Nicotiana benthamiana.

A minireplicon derived from a Closteroviridae virus is a polynucleotide, comprising a nucleic acid sequence encoding only proteins, each of which corresponds to a natural viral replication protein of the Closteroviridae virus required for replication of the virus. Each encoded protein exhibits the same function as its corresponding natural viral replication protein, and may have an amino acid sequence at least about 80%, 85%, 90%, 95%, or 99%, preferably at least about 95%, more preferably at least about 99%, most preferably 100%, identical to that of its corresponding natural viral replication protein. The minireplicon may be generated from the genome of the Closteroviridae virus by deleting nucleic acid sequences, including genes, not required for the replication of the virus. For example, a BYV minireplicon may be a replication gene block formed by the L-Pro domain, methyltransferase (Met), helicase (Hel) and RNA-dependent RNA polymerase (Pol) as shown in FIG. 1.

For each nucleic acid molecule of the present invention, a vector comprising the nucleic acid is provided. The vector may include border sequences of a bacterial transfer DNA at either end, and be situated in a bacterial transfer DNA, to allow for delivery of the nucleic acid of the present invention into a plant cell. Specifically, the vector may comprise one or more nucleic acid sequences derived from a Ti plasmid of a binary vector (e.g., right border (RB) and left border (LB) in FIG. 2). Such a vector, including elements of a Ti plasmid and a viral vector, is also called a launch vector. This vector may also be used for co-expression of a target polynucleotide of interest with a protein, such as a silencing suppressor or a modifying enzyme such as PNGaseF, to modify, affect expression and/or increase production the target polypeptide. The protein may facilitate maturation or accumulation of the target polypeptide.

A heterologous polynucleotide is a polynucleotide that is foreign, not native, to the Closteroviridae virus and the target cell. It may comprise a nucleic acid sequence encoding a target polypeptide, which may be expressed in a plant cell.

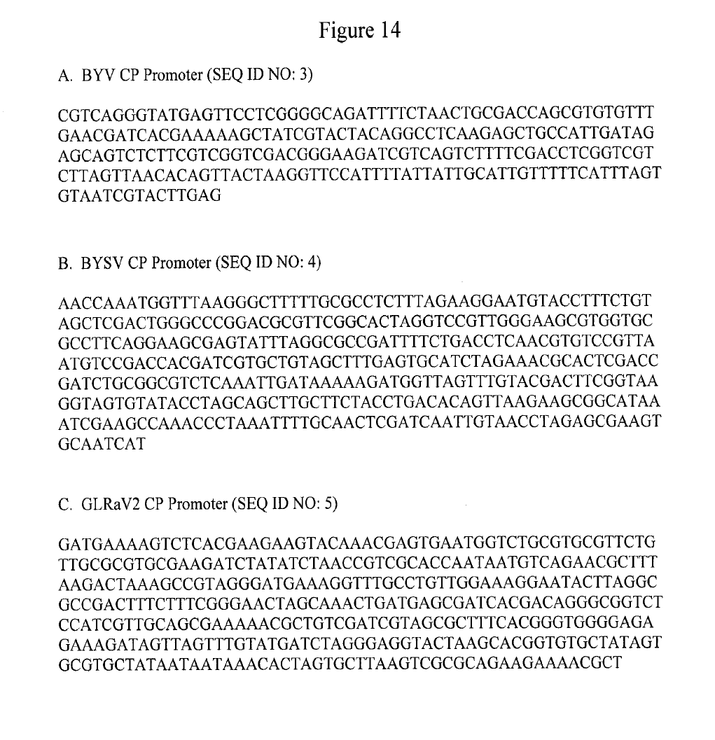

In the nucleic acid molecule of the present invention, each heterologous polynucleotide encodes a target polypeptide, and may be operatively linked to a viral promoter derived from any virus in the family Closteroviridae. Examples of suitable viral promoters include BYV promoters, Grapevine leafroll-associated virus 2 (GLRaV2) promoters, and/or Beet Yellows stunt virus (BYSV) promoters. Preferably, polynucleotides in the same nucleic acid molecule are operably linked to different viral promoters. Exemplary viral promoters include BYV CP promoter (SEQ ID NO: 3; FIG. 14), BYSV CP promoter (SEQ ID NO: 4; FIG. 14), and GLRaV2 CP promoter (SEQ ID NO: 5; FIG. 14).

The nucleic acid molecule of the present invention may further comprise a polynucleotide encoding one or more movement proteins derived from the Closteroviridae virus. Examples of the movement proteins include p6, Hsp70h, p64, CPm, CP and p20 of BYV. The movement proteins may enhance the movement of the nucleic acid molecule from one plant cell to another and cause systemic spread of the nucleic acid molecule, thereby increasing plant production level of the target polypeptide encoded by the heterologous polynucleotide by, for example, at least about 1%, 5%, 10%, 15%, 20%, 30%, 40%, 50%, 60%, 70%, 80%, 90%, 95%, 100%, 200%, 500% or 1000%.

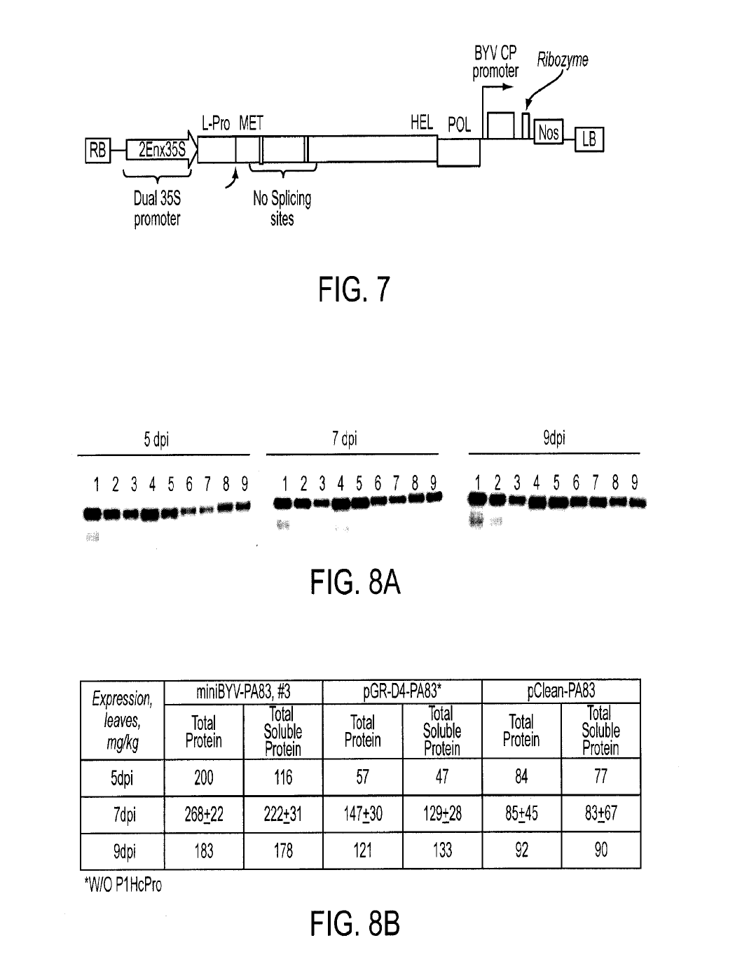

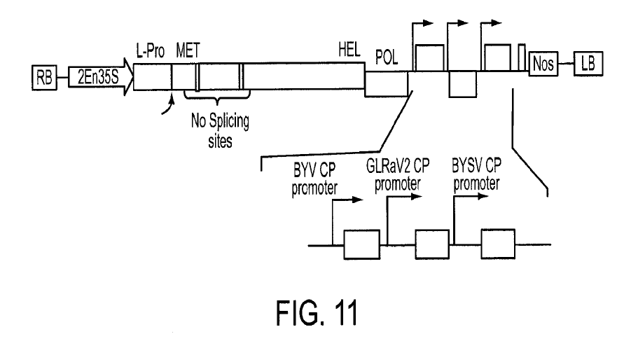

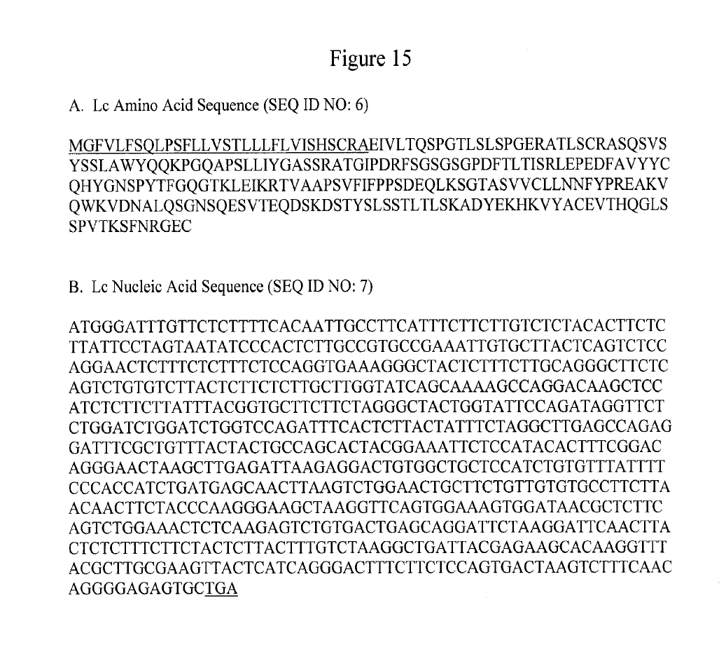

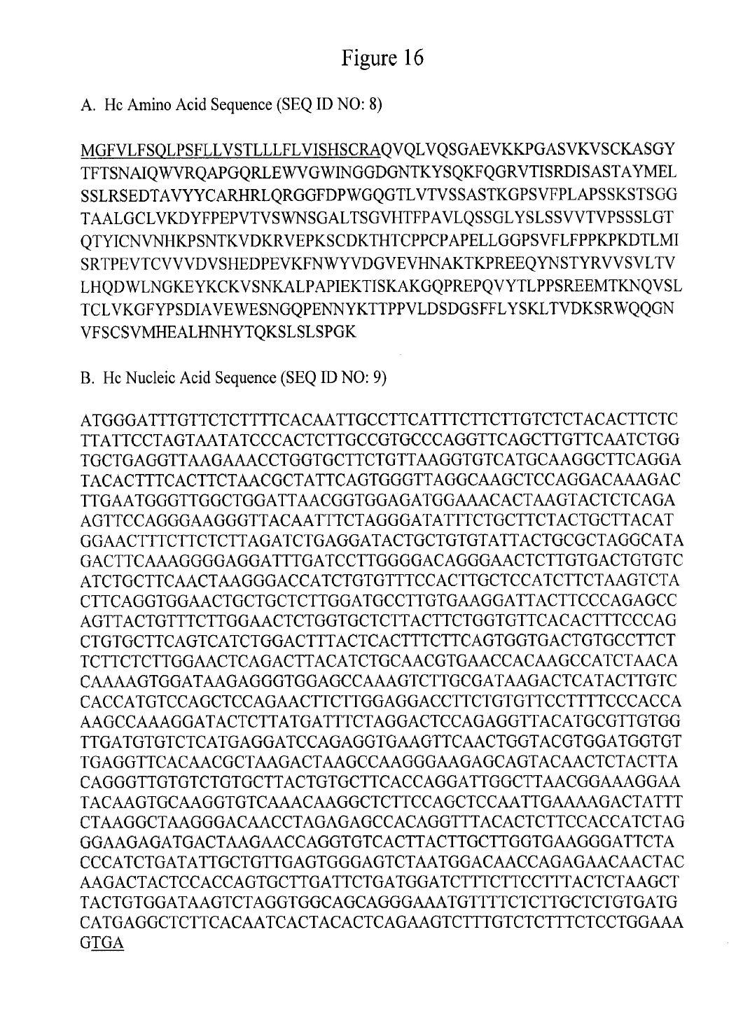

The nucleic acid molecule of the present invention may comprise one (e.g., FIG. 7), two (e.g., FIGS. 2, 4, 5, and 9), three (e.g., FIG. 11) or more heterologous polynucleotides, each of which encodes a target polypeptide. Preferably, the nucleic acid molecule comprises two or more heterologous polynucleotides encoding two or more target polypeptides, and the target polypeptides are expressed from the same minireplicon of the nucleic acid molecule within a plant cell. The target polypeptide may constitute a subunit of a protein. The target polypeptides may be capable of forming a protein such as an enzyme or antibody. For example, the nucleic acid molecule may comprise two heterologous polynucleotides encoding Hc (FIG. 16) and Lc (FIG. 15) of an antibody. In some embodiments, the nucleic acid may encode two target polypeptides, in which a first target polypeptide is capable of modifying, affecting expression and/or increasing production of a second target polypeptide in a plant cell. The first target polypeptide may facilitate maturation of the second polypeptide, which becomes biologically active. The first target polypeptide may also facilitate accumulation of the second polypeptide.

A target polypeptide may be any polypeptide capable of forming or becoming a functional protein (e.g., an enzyme or antibody) or a vaccine candidate. A target polypeptide may be of any size. It may have at least about 6, 10, 50, 100, 200, 300, 400, 500, 750, or 1000 amino acids, preferably at least about 100 amino acids, more preferably at least about 500 amino acids, most preferably at least 750 amino acids. It may also be at least about 10, 20, 50, 75, 100, 125, 150, or 200 kD, preferably at least about 100 kD, more preferably at least about 125 kD, most preferably at least about 150 kD.

A target polypeptide may be immunogenic. It may comprise one or more epitopes (linear and/or conformational) that are capable of stimulating the immune system of a subject to make a humoral and/or cellular antigen-specific immune response. A humoral immune response refers to an immune response mediated by antibodies produced by B lymphocytes, or B cells, while a cellular immune response refers to an immune response mediated by T lymphocytes, or T cells, and/or other white blood cells. In general, a B-cell epitope contains at least about 5 amino acids but can be 3-4 amino acids, while a T-cell epitope includes at least about 7-9 amino acids and a helper T-cell epitope includes at least 12-20 amino acids. A target polypeptide may be derived from a protein (e.g., a surface protein or toxin subunit) of a pathogenic organism or pathogen.

A “subject” may be an animal. For example, the animal may be an agricultural animal (e.g., horse, cow and chicken) or a pet (e.g., dog and cat). Preferably, the subject is a mammal. Most preferably, the subject is a human. The subject may be a male or female. The subject may also be a newborn, child or adult. The subject may have suffered a disease or medical condition.

For each of the nucleic acid molecules of the present invention, a method for producing one, two, three or more target polypeptides in a plant cell is provided. The method comprises (a) introducing the nucleic acid molecule into a plant cell; and (b) maintaining the plant cell under conditions permitting production of the target polypeptide(s) in the plant cell. The nucleic acid molecule comprises a minireplicon derived from a Closteroviridae virus, and is capable of replicating in the plant cell. The nucleic acid molecule further comprises one, two, three or more heterologous polynucleotides, each of which encodes a target polypeptide. The plant cell may be a cell in a plant, a plant part (e.g., leaf, stem, root, floral tissue, seed or petiole) or a cell culture medium. The plant may be a whole growing plant. Preferably, the plant cell is in a plant leaf.

The nucleic acid molecule of the present invention may be introduced into a plant cell using techniques known in the art. For example, the nucleic acid molecule may be delivered into the plant cell via infiltration, bombardment, or manual inoculation. The nucleic acid molecule could be used as a part of an inducible system activated by, for example, chemical, light or heat shock. Preferably, the nucleic acid molecule is introduced into a plant cell via infiltration.

For production of a target polypeptide by a plant or plant cells infected by a vector of the present invention, the infected plant or plant cells are maintained under conditions permitting for the production. Such conditions include suitable temperature, humidity, pressure, timing, and illumination. As described below in Examples 2 and 4-6, nucleic acid molecules of the present invention have been introduced into plant cells, which were maintained under conditions permitting production of one or two polypeptides, and such production was observed. The production method may further comprise purifying at least one of the target polypeptide(s) from the plant. The target polypeptide may be purified from the plant using techniques known in the art. For example, the target polypeptide may be purified from the plant using an antibody or a receptor capable of binding the target polypeptide. The purification process may comprise extraction of the target from N. benthamiana using extraction buffer. After low speed centrifugation, supernatant may be clarified by filtration and used for chromatography. The purified product may be at least about 10%, 20%, 30%, 40%, 50%, 60%, 70%, 80%, 90%, 95% or 99%, preferably at least about 50%, more preferably at least about 75%, most preferably at least about 95%, pure. The target polypeptide may be used in a crude plant extract. For example, an industrial enzyme expressed in plant tissues according to the present invention, and a crude plant extract containing the enzyme may be used in an industrial process.

In the production method according to the present invention, the Closteroviridae virus may be any virus in the family of Closteroviridae. Examples of the Closteroviridae virus include BYV, Grapevine leafroll-associated virus 2 (GLRaV2), and Beet Yellows stunt virus (BYSV). Preferably, the Closteroviridae virus is BYV. The vector may further comprise a polynucleotide encoding one or more movement proteins derived from the Closteroviridae virus. The movement proteins may enhance movement of the heterologous polynucleotide from one plant cell to another plant cell, and thereby increase plant production level of the target polypeptide encoded by the heterologous polynucleotide by, for example, at least about 1%, 5%, 10%, 15%, 20%, 30%, 40%, 50%, 60%, 70%, 80%, 90%, 95%, 100%, 200%, 500% or 1000%.

For each production method of the present invention, a composition comprising the one, two, three or more target polypeptides produced thereby is provided. Also provided is a method of treating a subject in need of the one, two, three or more target polypeptides. The treatment method comprises administering to the subject an effective amount of a pharmaceutical composition comprising the target polypeptide(s). The pharmaceutical composition may further comprise a pharmaceutically acceptable carrier or diluents. Suitable carriers or diluents are known in the art and include, but are not limited to, saline, buffered saline, mannitol, L-histidine, polysorbate 80, dextrose, water, glycerol, ethanol, and combinations thereof. The pharmaceutical composition may optionally contain an adjuvant. The pharmaceutical composition may have a pH of about 4.0-10.0, preferably 5.6-7.0.

The term “an effective amount” refers to an amount of a pharmaceutical composition comprising the target polypeptide(s) required to achieve a stated goal (e.g., treating a subject in need of the target polypeptide(s), or inducing an immune response in a subject). The effective amount of the pharmaceutical composition comprising the target polypeptide(s) may vary depending upon the stated goal, the physical characteristics of the subject, the nature and severity of the need of the target polypeptide(s), the existence of related or unrelated medical conditions, the nature of the target polypeptide(s), the composition comprising the target polypeptide(s), the means of administering the composition to the subject, and the administration route. A specific dose for a given subject may generally be set by the judgment of a physician. The pharmaceutical composition may be administered to the subject in one or multiple doses.

The target polypeptide(s) may be formulated in a pharmaceutical composition of the present invention. The pharmaceutical composition may be formulated for administration to a subject via various routes, for example, oral, sublingual, intranasal, intraocular, rectal, transdermal, mucosal, topical or parenteral administration.

In a production method according to the present invention, the one, two, three or more target polypeptides may be one or more subunits of a protein, and the maintaining conditions may further permit production of the protein in the plant cell. A composition comprising the protein produced thereby is provided. A method of treating a subject in need of the protein is also provided. The treatment method comprises administering to the subject an effective amount of a pharmaceutical composition comprising the protein. The protein may be an enzyme.

In a production method according to the present invention, a first target polypeptide may be capable of modifying, affecting expression, and/or increasing production of a second target polypeptide in the plant cell, and the maintaining conditions may further permit modifying, affecting expression, and/or increasing production of the second target polypeptide by the first target polypeptide in the plant cell. A composition comprising the second target polypeptide produced thereby is provided. A method of treating a subject in need of the modified target polypeptide is also provided. The treatment method comprises administering to the subject an effective amount of a pharmaceutical composition comprising the polypeptide.

In a production method according to the present invention, the target polypeptides may be Hc and Lc of an antibody, and the maintaining conditions may further permit production of the antibody in the plant cell. A composition comprising the antibody produced thereby is provided. A method of treating a subject in need of the antibody is also provided. The treatment method comprises administering to the subject an effective amount of a pharmaceutical composition comprising the antibody.

In a production method according to the present invention, the target polypeptide may be immunogenic. A composition comprising the immunogenic target polypeptide is provided. A method for inducing an immune response in a subject is also provided. The immunogenic method comprises administering to the subject an effective amount of a pharmaceutical composition comprising the immunogenic target polypeptide. Where the immunogenic target polypeptide is derived from a pathogen, the immunogenic method may be used for inducing a protective immune response against the pathogen in a subject by administering to the subject an effective amount of a pharmaceutical composition comprising the immunogenic target polypeptide. The pathogen may be an intracellular or extracellular pathogen.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

All documents, books, manuals, papers, patents, published patent applications, guides, abstracts, and other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

### Example 1

### Construction of BYV-Based Launch Vector for Monoclonal Antibody Expression

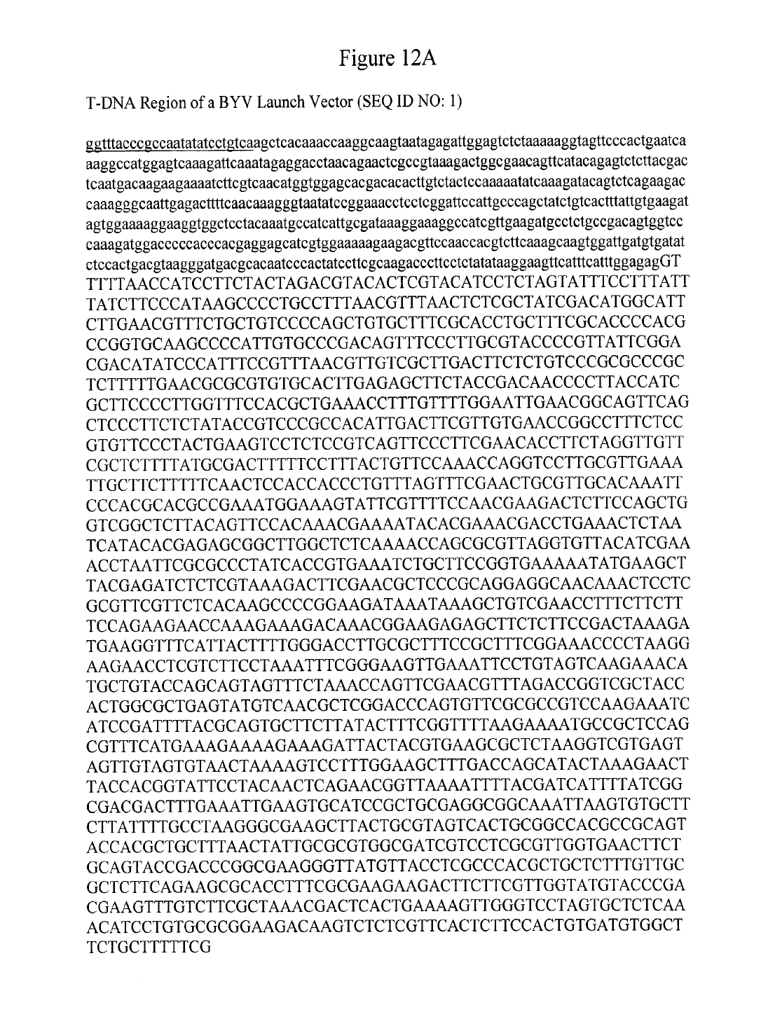

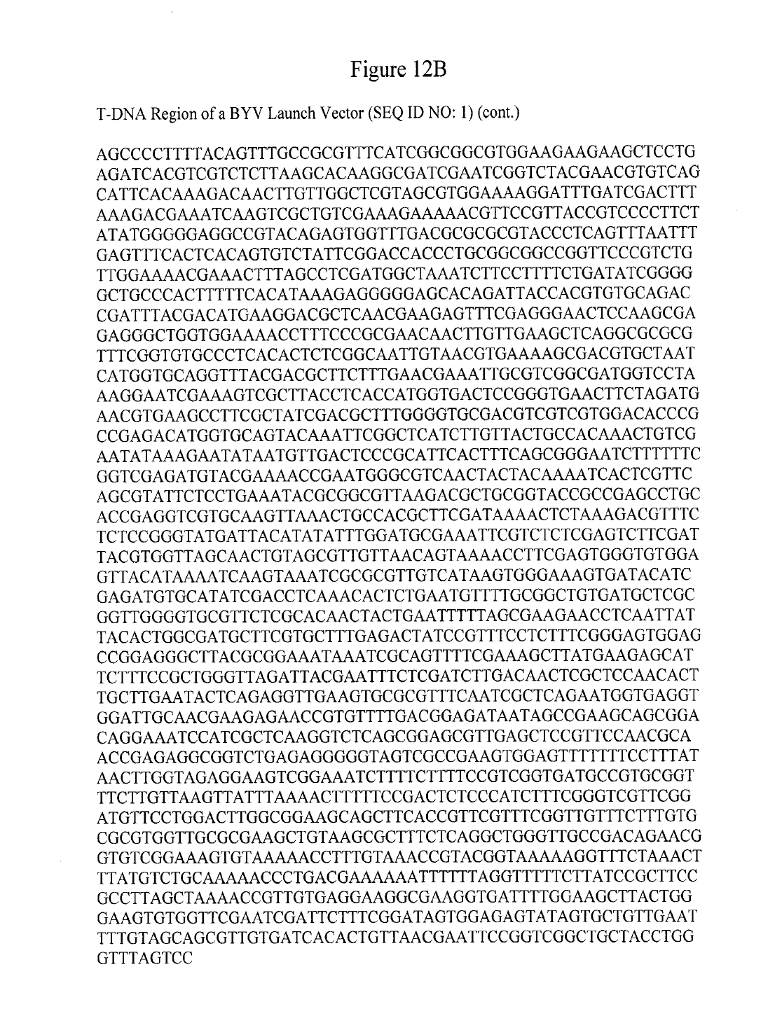

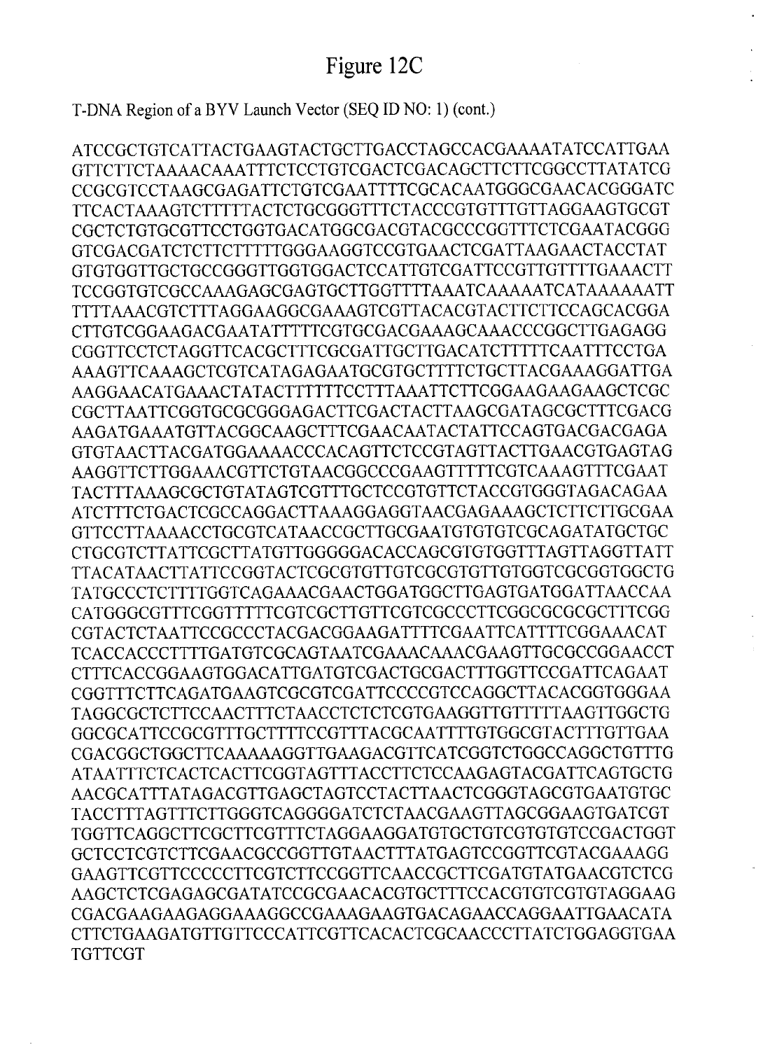

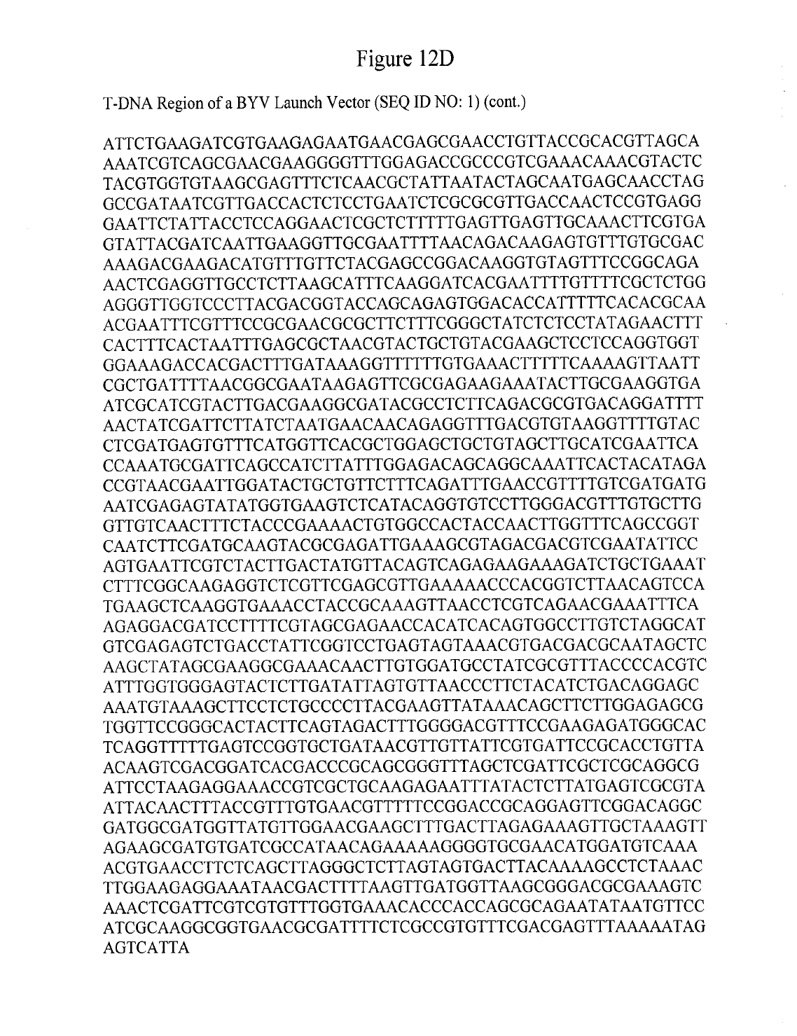

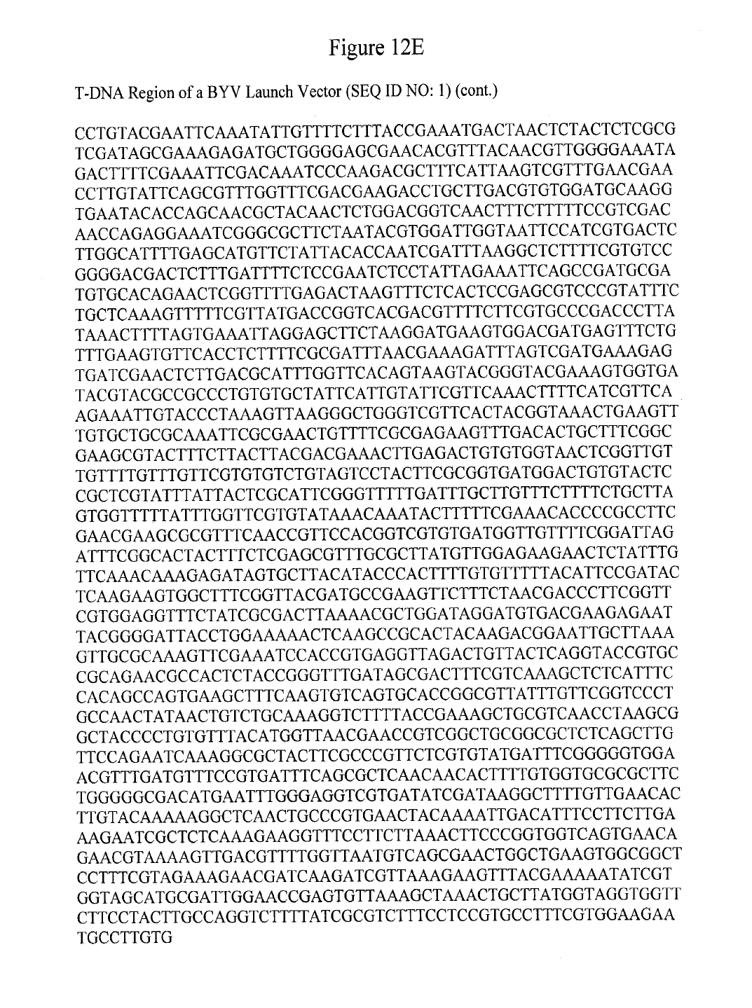

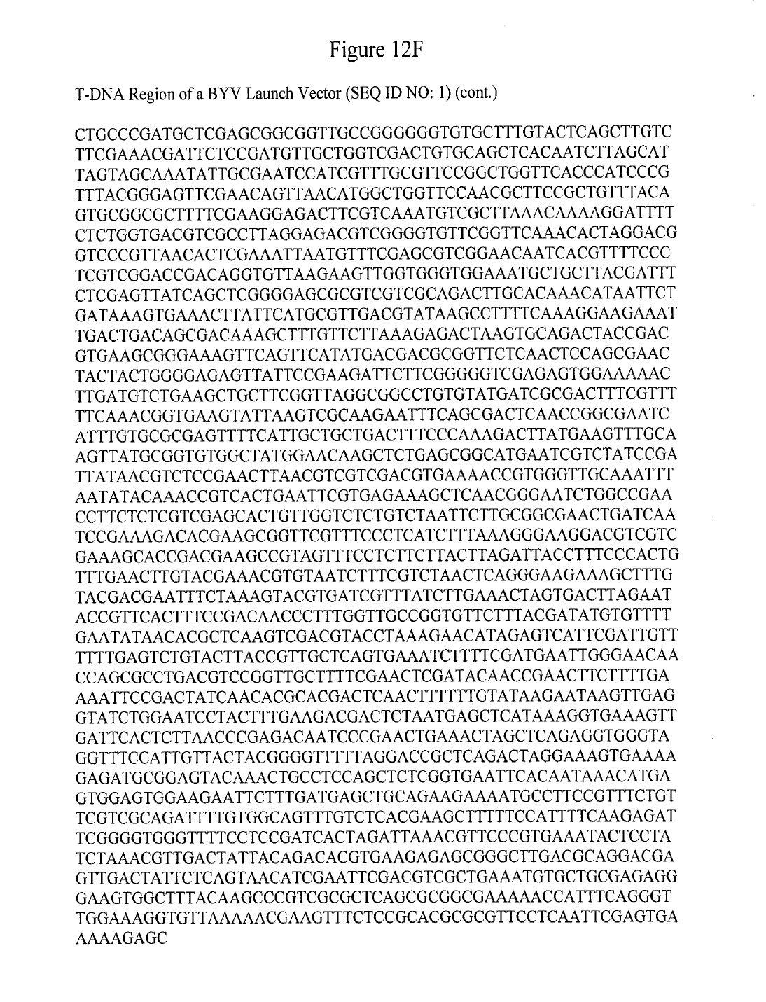

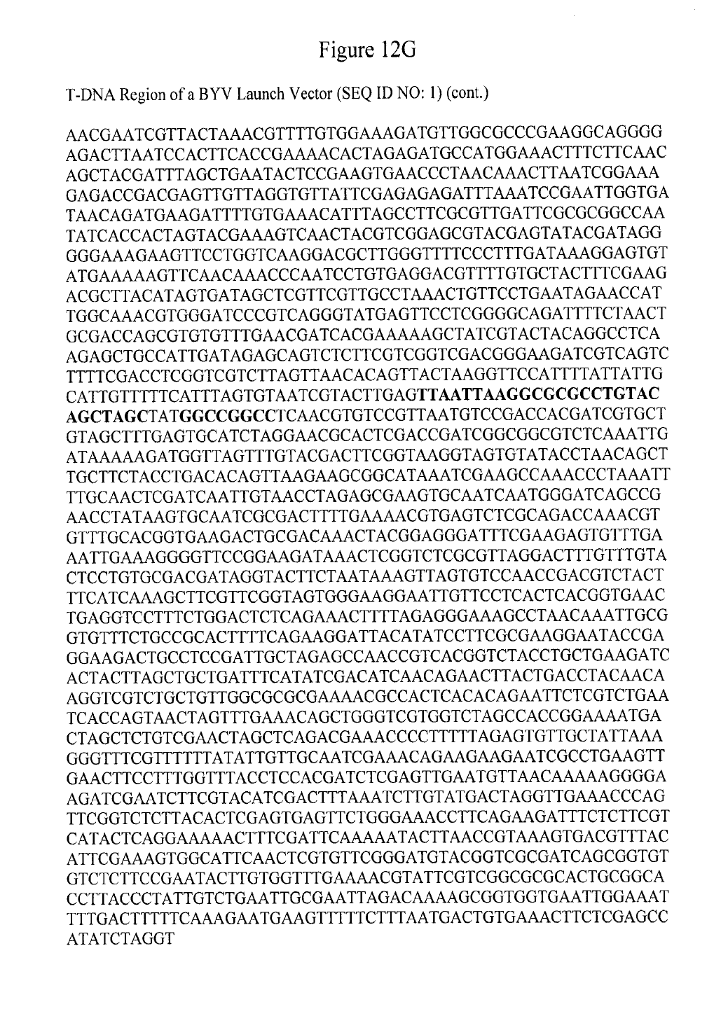

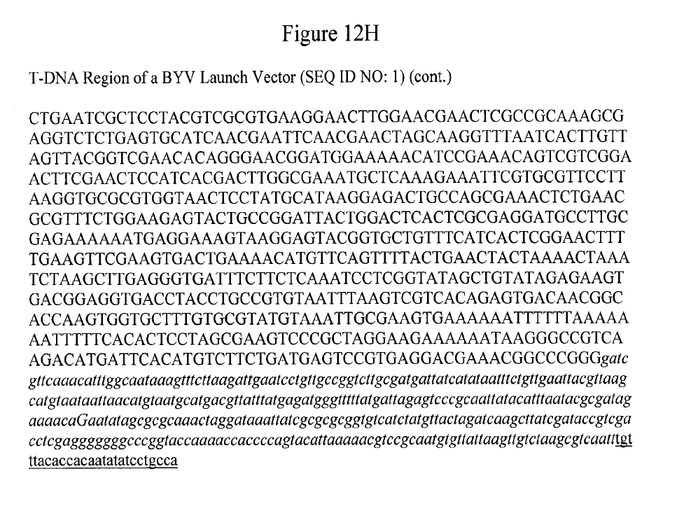

A BYV-based launch vector for simultaneous expression of two target polypeptides within the same host cell was constructed (FIG. 2). The BYV launch vector was used as a carrier for expression of two ORFs, Hc and Lc of a mAb against PA of anthrax. For cloning of two foreign genes (Lc and Hc of the anti-PA mAb), a multiple cloning site (MCS) was introduced into the BYV genome between the CPm and CP coding sequences (SEQ ID NO: 1, FIG. 12). The MCS contains 5 restriction sites, PacI/AscI/BsrGI/NheI/FseI, in addition to the native BamHI restriction site. After inserting the MCS, two heterologous closteroviral CP promoters were introduced into the BYV genome: the GLRaV2 CP promoter and the BYSV CP promoter (FIG. 2). As a result, the sequences of Hc (SEQ ID NO: 9; FIG. 16) and Lc (SEQ ID NO: 7; FIG. 15) of the anti-PA mAb were cloned under the control of the BYV CP and the GLRaV2 CP promoters, respectively. Meanwhile, the BYSV CP promoter drives the BYV CP (FIG. 2). The resulting construct pCB-BYV-PA-HcLc was transformed into Agrobacterium tumefaciens strain GV3101.

### Example 2

### Expression of Anti-PA mAb in Systemically BYV-Infected Leaves

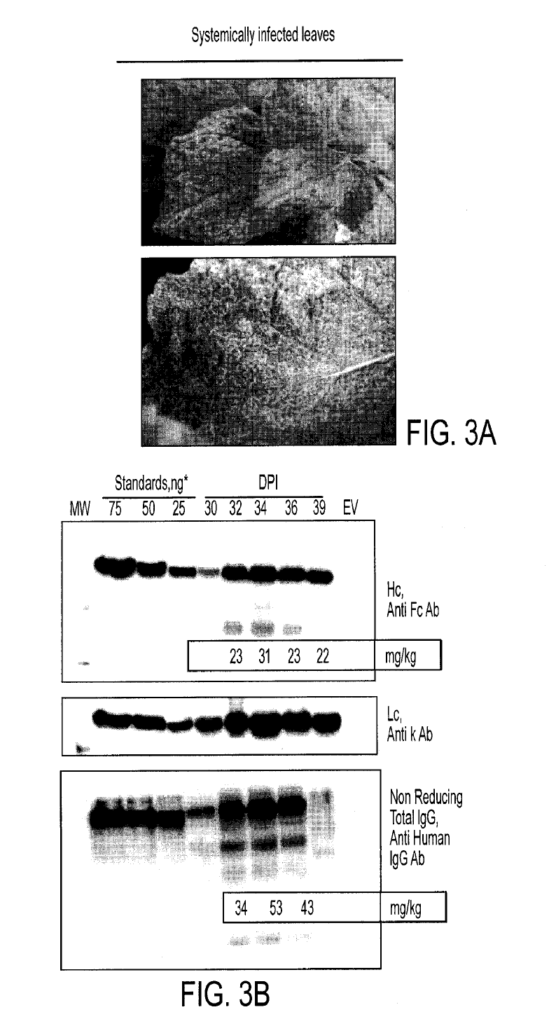

To confirm the stability of the virus and the expression and assembly of the anti-PA mAb, 5-week-old N. benthamiana leaves were manually co-infiltrated by overnight-grown (at 28° C.) cultures of agrobacteria carrying a BYV vector encoding Hc and Lc of the anti-PA mAb and agrobacteria carrying a binary vector encoding a silencing suppressor P1HcPro from Turnip mosaic virus (Kasschau et al, 2003), at a ratio of 1.0:0.2 OD₆₀₀. After 30 dpi, systemic symptoms of the BW infection were observed (FIG. 3A). In particular, the infected leaves showed clearing veins as the systemic symptoms. Samples were collected from systemically infected leaves at 30, 32, 34, 36 and 39 dpi.

To demonstrate the expression of Lc and Hc of the anti-PA mAb, Western blot analysis was employed (FIG. 3B). To assess the Hc and Lc expression levels, horseradish peroxidase (HRP)-conjugated goat anti-human Hc and Lc antibodies (Bethyl Laboratories Inc.) were used at dilution of 1:5000 and 1:2000, respectively. A purified anti-PA mAb was used to serve as a positive control. The expression levels were calculated using GeneGnome5 gel imaging and analysis systems from Synoptics Ltd. Using the same technique under non-reducing conditions, the expression level of the assembled anti-PA mAb was calculated. The maximum expression level was determined to be 53 mg/kg of fresh leaf weight at 34 dpi.

### Example 3

### Construction of a T-DNA-Based BYV Minireplicon

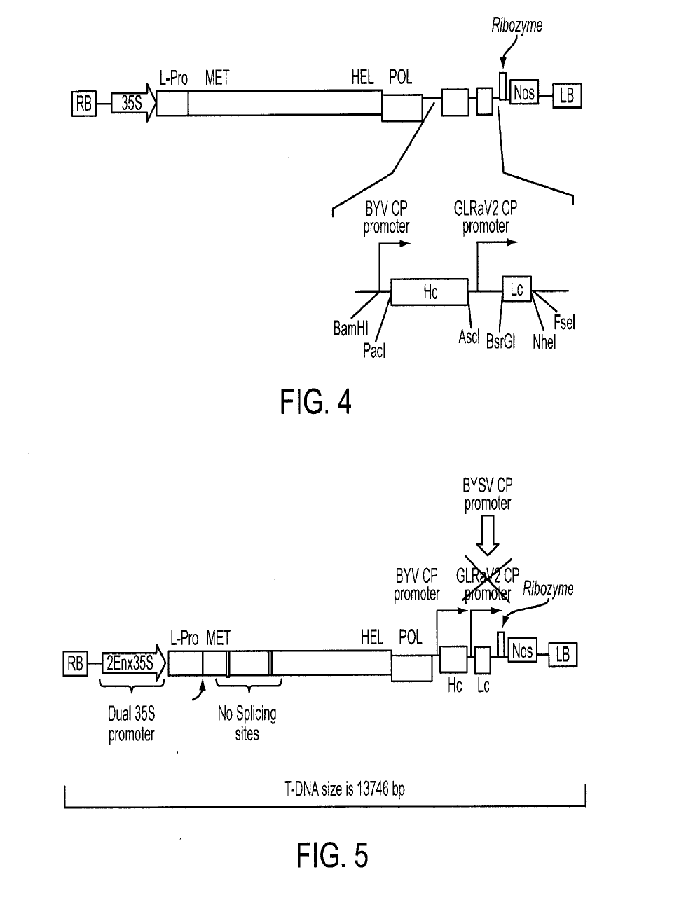

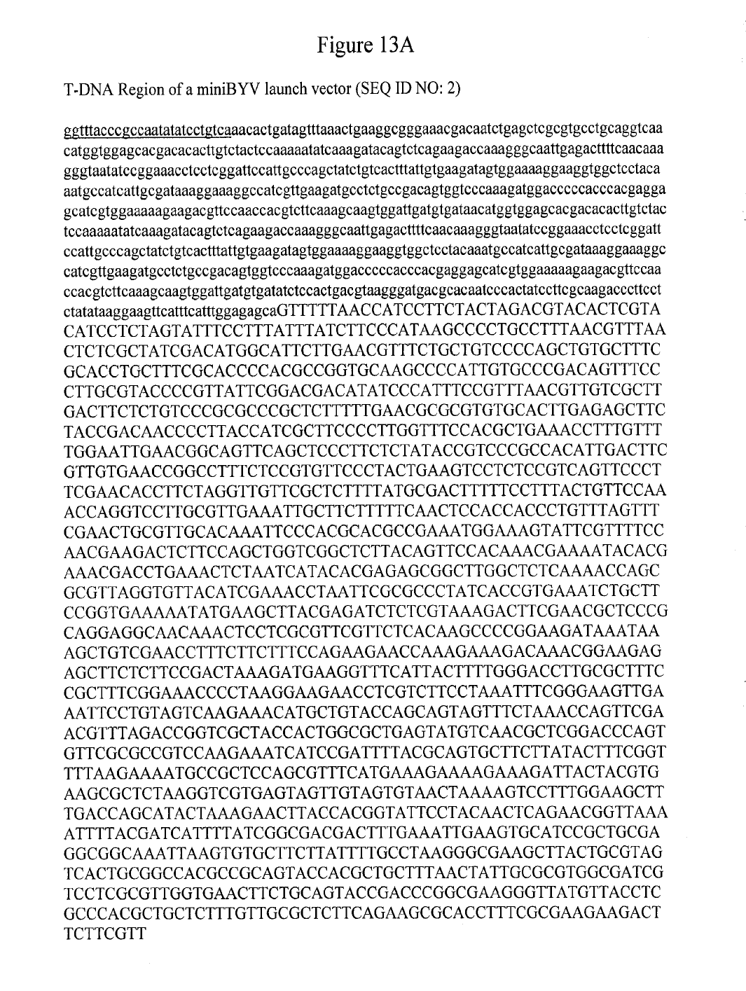

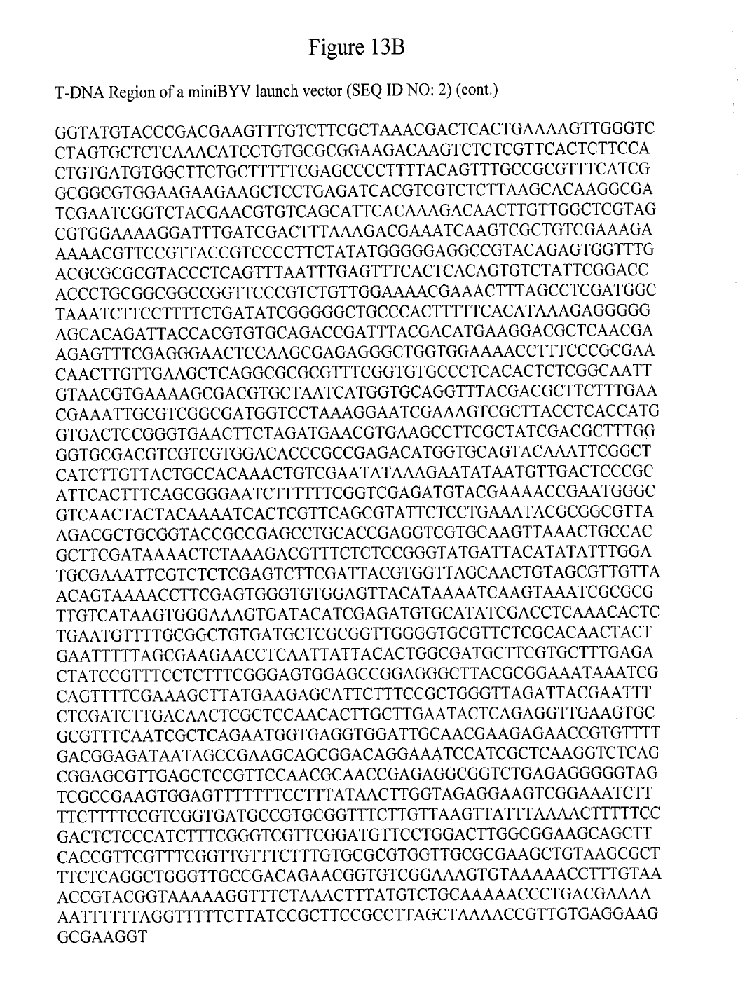

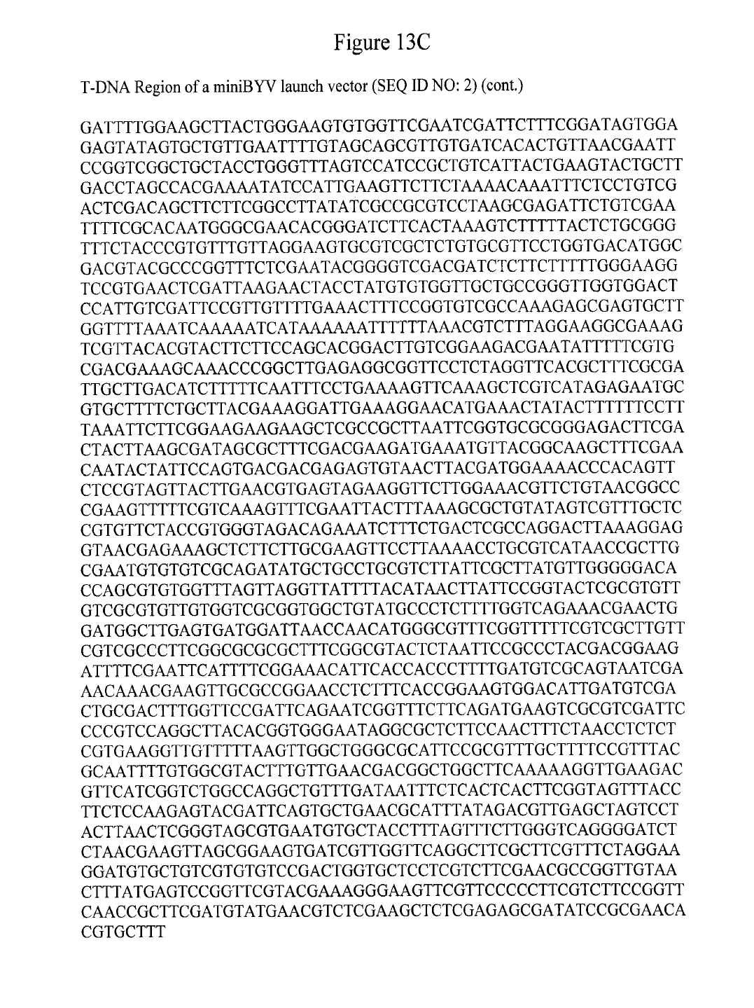

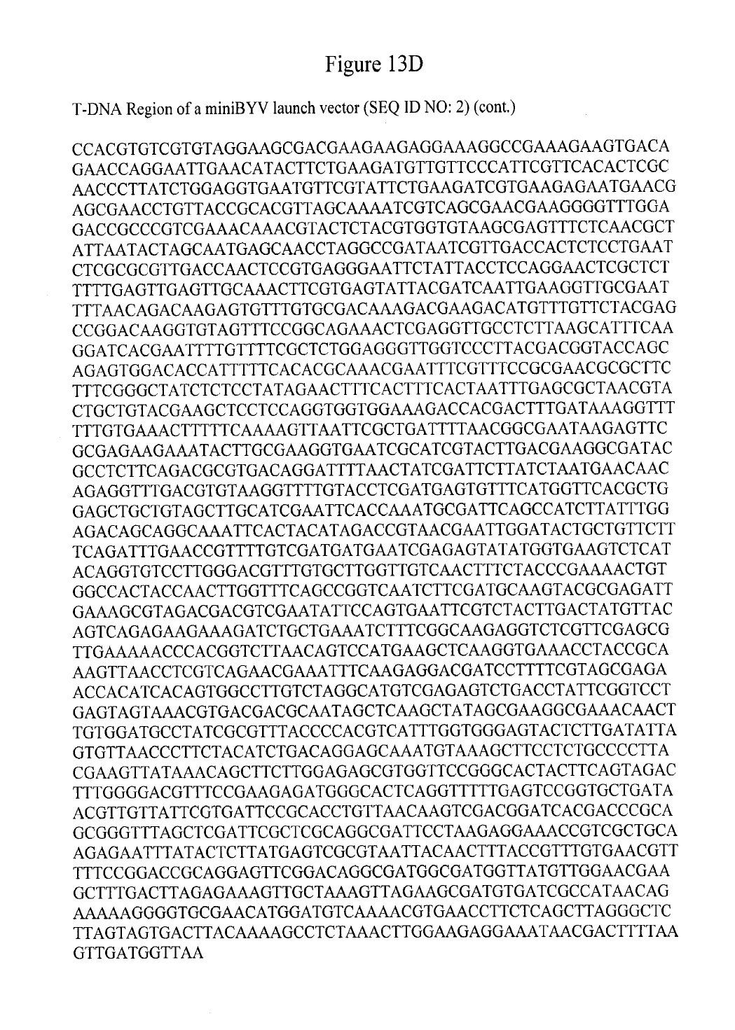

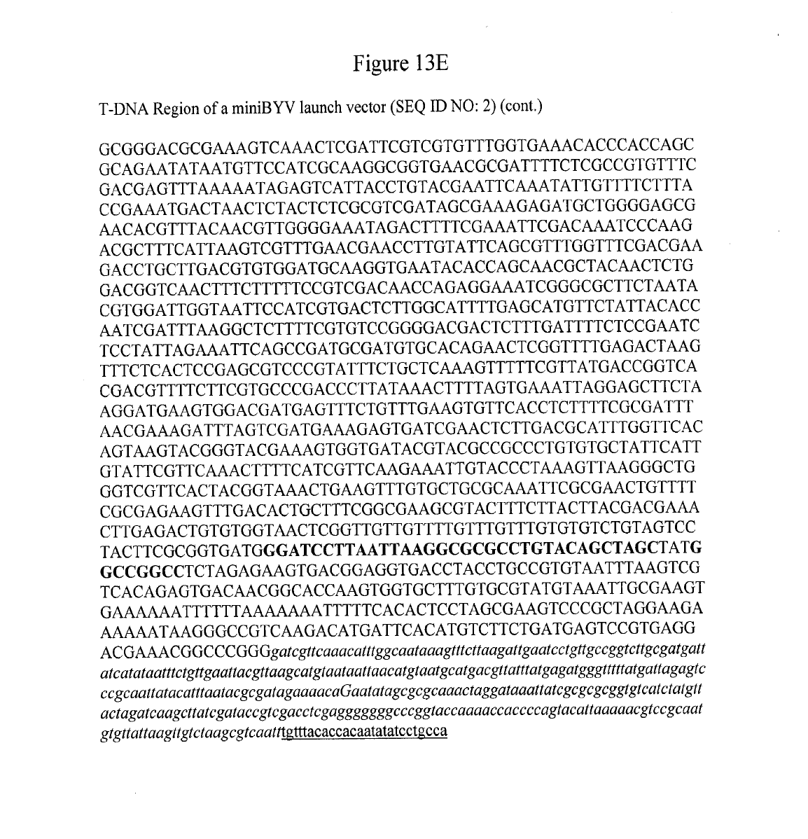

To decrease the production time and increase the antibody expression level, a T-DNA-based BYV minireplicon (miniBYV) was engineered by removing all genes which are not necessary for viral replication from the BYV-based launch vector as described in Example 1 (FIGS. 1, 2 and 4). Using the native BamHI restriction site, the same MCS as the one inserted into the whole-length BYV-based vector in Example 1 was introduced into the miniBYV replicon (SEQ ID NO: 2; FIG. 13) (FIGS. 2 and 4). This strategy allowed for using heterologous closteroviral subgenomic promoters to express two foreign genes from a single miniBYV replicon. In addition, two closteroviral promoters, the BYV CP promoter and GLRaV2 CP promoter, were introduced to drive Hc (SEQ ID NO: 9; FIG. 16) and Lc (SEQ ID NO: 7; FIG. 15) of the anti-PA mAb, respectively.

To prevent splicing and increase the efficiency of viral invasiveness, the canonical splicing sites were removed from the viral replicase sequence. To increase the transcription level of miniBYV RNA, Cauliflower mosaic virus 35S promoter with a dual enhancer was inserted upstream of the 5′ end of the miniBYV sequence (FIG. 4).

To increase the amount of the synthesized initial transcript, the 35S promoter with dual enhancers was introduced upstream of the miniBYV sequence to generate a modified miniBYV vector (FIG. 5). The weak GLRaV2 CP promoter was replaced by the strong BYSV CP promoter. The BYV replicase was analyzed for the presence of the canonical splicing sites using the SplicePredictor software from the Center for Bioinformatics and Biological Statistics, Iowa State University. Arabidopsis was used as a splicing site model. To avoid potential splicing, the high-scoring canonical acceptor splicing site within the BYV replicase was mutated by substituting the nucleotide 2219 (GenBank Accession No. AF 190581) from A to C, which was confirmed by sequencing. This also allowed for knocking out the donor site in the position 3606. The final size of T-DNA insert carrying miniBYV with Hc and Lc of the mAb was 13,746 bp (FIG. 5).

### Example 4

### Expression of Anti-PA mAb in Leaves Systemically Infected with the Modified miniBYV vector (pCB-BYV-Hc-Lc)

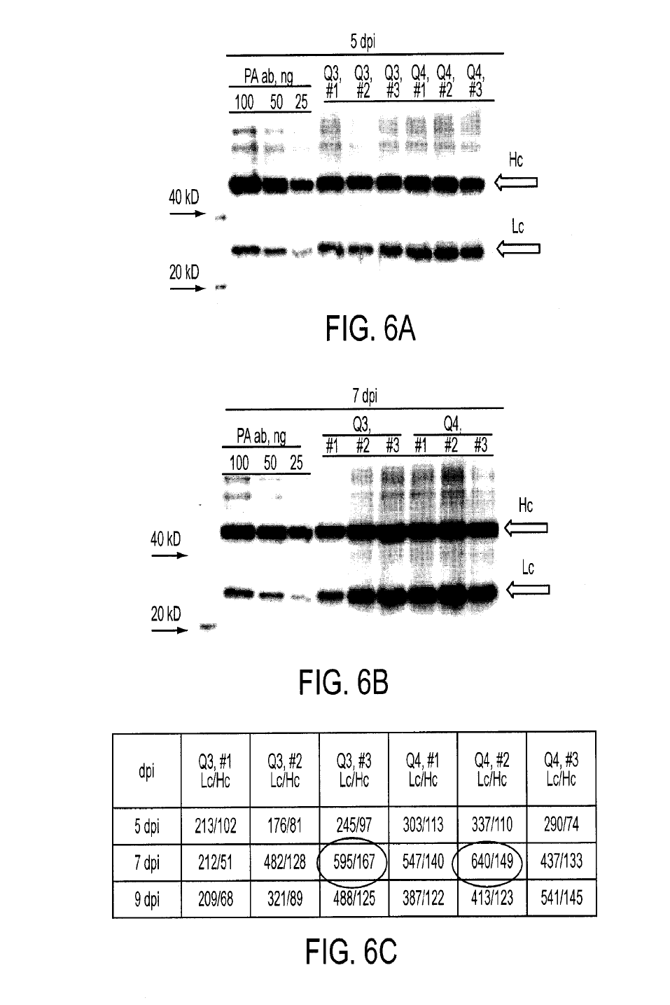

To examine the expression level of Hc and Lc of the mAb from the modified miniBYV vector, 5-week-old N. benthamiana plants were infiltrated as described in Example 2 using two clones (Q3 and Q4, confirmed by sequencing) and three agro colonies were collected for each clone. To confirm that both Hc and Lc were expressed, the leaf disks were taken at 5, 7 and 9 dpi, analyzed by Western blotting and calculated as described above. The results demonstrate a three-fold increase in the expression level of Hc and Lc of the anti-PA mAb using the modified miniBYV launch vector carrying two strong promoters (FIG. 6) and suggest that the miniBYV vector can be used for antibody production.

### Example 5

### Expression of a Large Protein (PA83) Using the miniBYV Vector

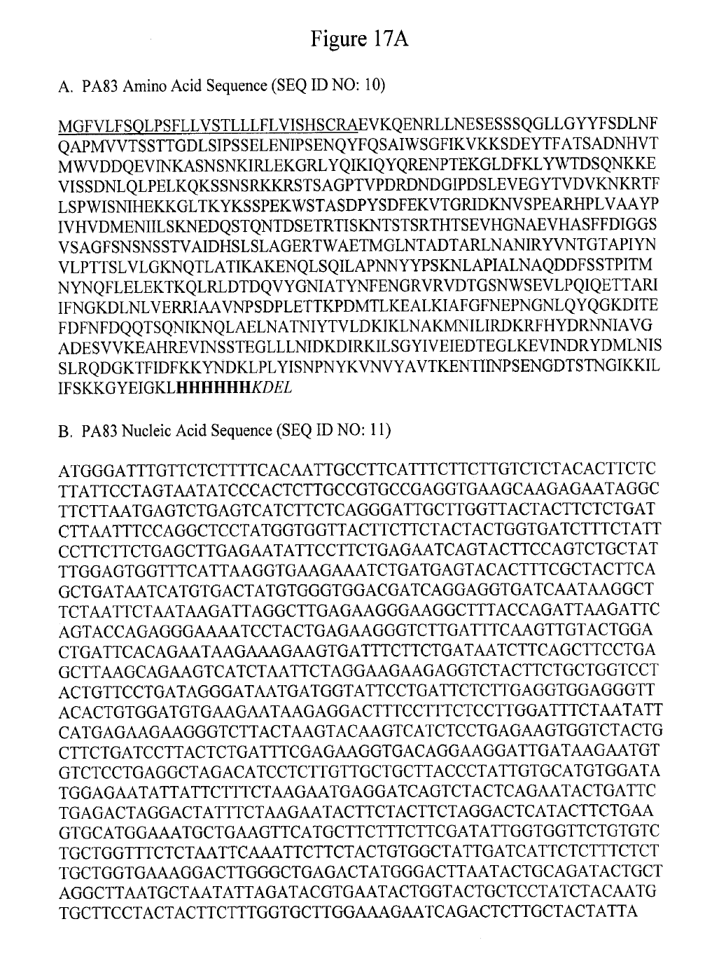

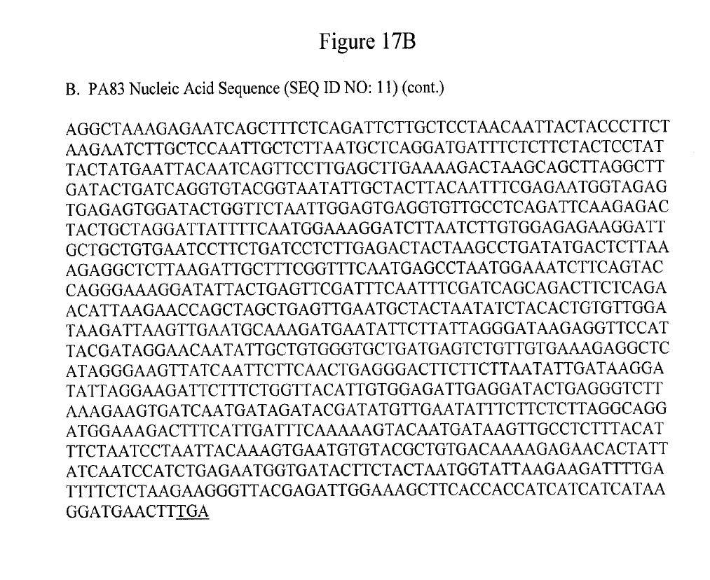

To investigate a possibility of using the miniBYV vector for expressing large proteins, anthrax Protective Antigen 83 (PA83, GenBank accession no. M22589) with a molecular weight of 83 kD from Bacillus anthracis was used. The sequence of PA83 with added PR-1a signal peptide from Nicotiana tabacum, 6× Histidine affinity purification tag and an endoplasmic reticulum (ER) retention signal (KDEL) (SEQ ID NO: 11; FIG. 17) was plant optimized by GENEART Inc. (Germany) and cloned into the miniBYV vector using PacI/NheI restriction sites (FIG. 7). The final construct was confirmed by sequencing. A binary vector carrying the miniBYV-PA83 vector was transformed into the GV3101 strain of agrobacteria. The expression level of PA83 from the miniBYV was compared to other vectors such as TMV-based launch vector pGR-D4-PA83 and a regular binary vector pClean283-PA83 carrying a dual 35S promoter with a TEV leader. Five-month-old N. benthamiana leaves were manually infiltrated as described above.

The total protein (TP) and total soluble protein (TSP) expression levels at 5, 7 and 9 dpi were analyzed (FIG. 8A). The infiltration and analysis were repeated three times for 7 dpi to confirm the calculated numbers. As shown in FIG. 8B, the highest expression level was observed for PA83 using the miniBYV vector at 7 dpi (268±22 mg/kg) with 83% solubility. The expression level was at least two times higher for the miniBYV replicon compared to the regular binary vector (pClean) and more than 60% higher compared to the TMV-based vector (D4) (FIG. 8).

Using an immobilized metal ion adsorption chromatography (IMAC) column, the PA83 protein expressed from the miniBYV replicon was purified. The purification process consisted of an extraction of the target from N. benthamiana leaf tissue with extraction buffer (50 mM Na-Phosphate pH 8.0, 500 mM NaCl, 20 mM Imidazole and 1 mM diethylcarbamic acid [DIECA]) at the 3:1 v/w ratio. After clarification, PA83 was captured using Ni-IMAC by employing the Chelating Sepharose Big Beads resin. The target was eluted with buffer contacting 300 mM Imidazole (obtained by mixing 60% of Buffer B [50 mM Na-Phosphate pH 7.5, 500 mM NaCl, 500 mM Imidazol] with 40% of Buffer A [50 mM Na-Phosphate pH 7.5, 500 mM NaCl]). Western blot analysis showed that final recovery of PA83 expressed from the miniBYV vector after IMAC purification was about 72%.

### Example 6

### Co-Expression of a Target Protein and an Enzyme Using the miniBYV Vector

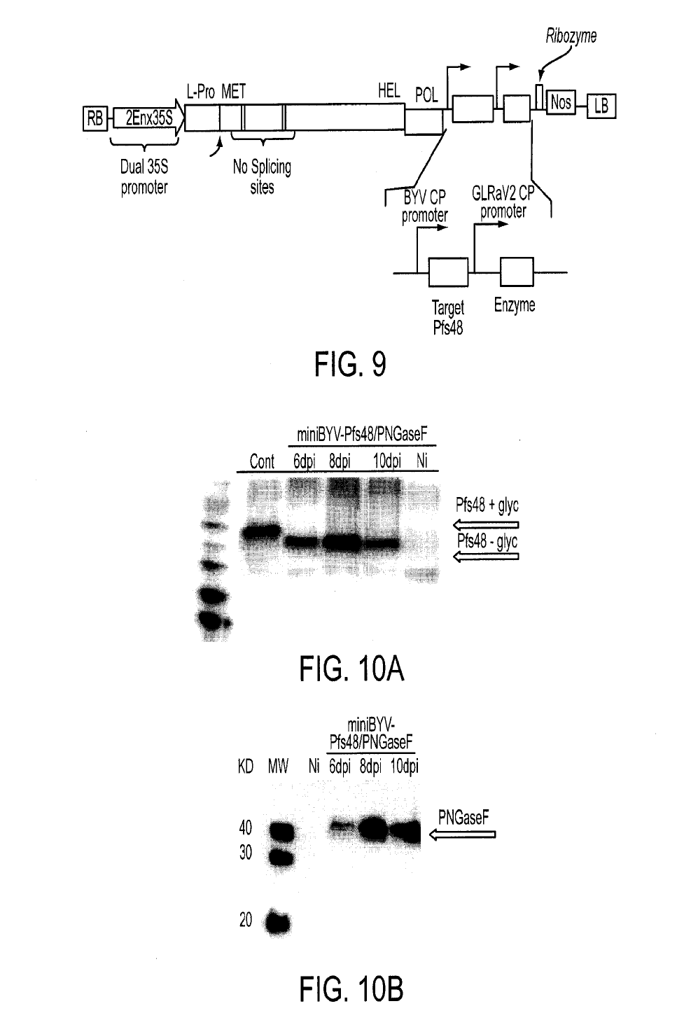

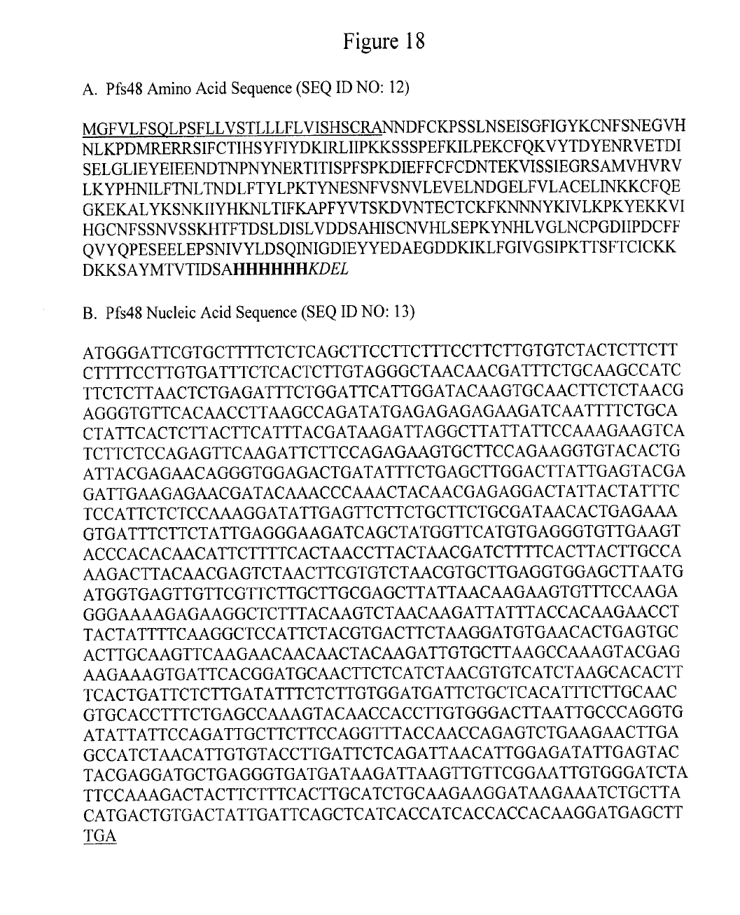

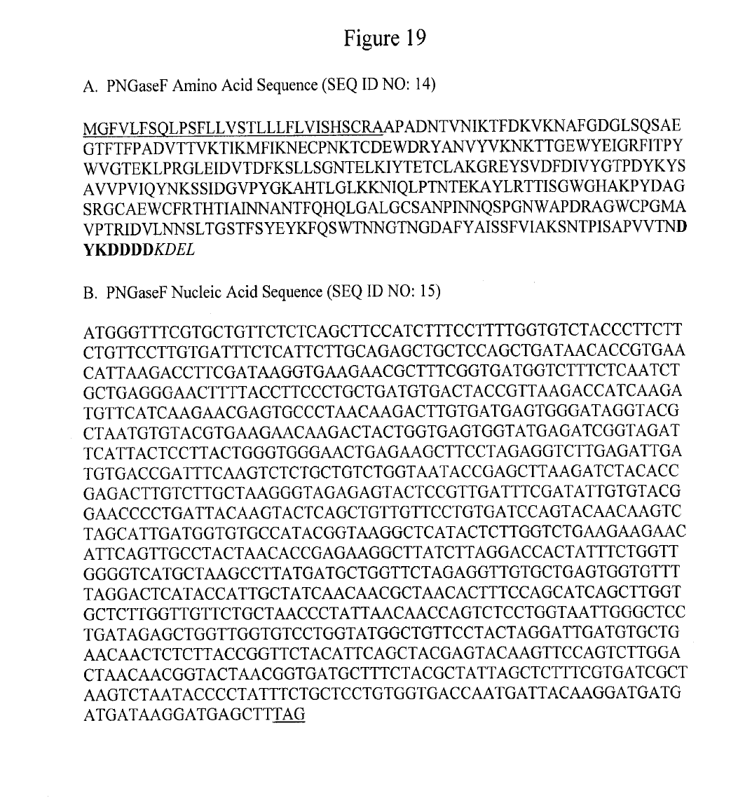

To explore a possibility of using the miniBYV vector for co-expression of a target protein and a modifying enzyme, malaria vaccine candidate protein Pfs48 (from protozoa parasite Plasmodium falciparum, accession # AAL74351) and endoglycosidase F from Elizabethkingia meningoseptica (PNGaseF, accession no AAA24932) were used. Again, a plant GENEART-optimized sequence of Pfs48 with the PR-1a peptide at the N-terminus and the 6×His-tag and KDEL on the C-terminus of the protein (SEQ ID NO: 13; FIG. 18) was used. For PNGaseF, the PR-1a peptide on N-terminus of the protein and FLAG-tag and KDEL ER retention signal on the C-terminus (SEQ ID NO: 15; FIG. 19) were used. Both sequences were cloned into the miniBYV vector as shown in FIG. 9. A weak GLRaV2 CP promoter was used to control PNGaseF because the enzyme toxic to the plant tissue when expressed at a higher level.

The expression level of Pfs48 was assessed using a mouse anti-histidine mAb at a 1:2000 dilution (anti-4×His, Qiagen Inc.). FIG. 10A shows that the electrophoretic mobility of the glycosylated Pfs48 (control, which was expressed w/o PNGaseF) was different (less mobility) compared to the non-glycosylated form of Pfs48 that was co-expressed with PNGaseF. The expression of PNGaseF was confirmed by Western blotting using a rabbit anti-FLAG primary mAb (Sigma) and a goat anti-rabbit secondary Ab (Biorad) (FIG. 10B). These results verify identical compartmentalization of both the target (Pfs48) and the enzyme (PNGaseF), which is probably inside of the virus replication complex. The results also verify that the PNGaseF acted on the Pfs48.

### REFERENCES

Dolja V V. Beet yellows virus: the importance of being different. Mol Plant Pathol. 2003 Mar. 1; 4(2):91-8.
Giritch A, Marillonnet S, Engler C, van Eldik G, Botterman J, Klimyuk V, Gleba Y. Rapid high-yield expression of full-size IgG antibodies in plants coinfected with noncompeting viral vectors. Proc Nati Acad Sci USA. 2006, 103(40):14701-6.
Gleba Y, Klimyuk V, Marillonnet S. Viral vectors for the expression of proteins in plants. Curr Opin Biotechnol. 2007, 18(2):134-41.
Hull A K, Criscuolo O, Mett V, Groen H, Steeman W, Westra H, Chapman G, Legutki B, Baillie L, Yusibov V. Human-derived, plant-produced monoclonal antibody for the treatment of anthrax. Vaccine. 2005, 18; 23(17-18):2082-6
Kasschau K D, Carrington J C. Long-distance movement and replication maintenance functions correlate with silencing suppression activity of potyviral HC-Pro. Virology. 2001, 285(1):71-81.
Mett V, Chichester J A, Stewart M L, Musiychuk K, Bi H, Reifsnyder C J, Hull A K, Albrecht M T, Goldman S, Baillie L W, Yusibov V. A non-glycosylated, plant-produced human monoclonal antibody against anthrax protective antigen protects mice and non-human primates from B. anthracis spore challenge. Hum Vaccin. 2011, 7 Suppl:183-90 Musiychuk K, Stephenson N, Bi H, Farrance C E, Orozovic G, Brodelius M, Brodelius P, Horsey A, Ugulava N, Shamloul A M, Mett V, Rabindran S, Streatfield S J, Yusibov V. A launch vector for the production of vaccine antigens in plants. Influenza Other Respi Viruses. 2007, 1(1):19-25
Peng C., Peremyslov V V, Mushegian A R, Dawson W O, Dolja V V. Functional specialization and evolution of leader proteinases in the family of Closteroviridae. J. Virol. 2001, 75(24):12153-60.
Prokhnevsky A I, Peremyslov V V, Napuli A J, Dolja V V. Interaction between long-distance transport factor and Hsp70-related movement protein of Beet yellows virus. 3 Virol. 2002, 76(21):11003-11.
Roy G, Weisburg S, Foy K, Rabindran S, Mett V, Yusibov V. Co-expression of multiple target proteins in plants from a tobacco mosaic virus vector using a combination of homologous and heterologous subgenomic promoters. Arch Virol. 2011, [Epub ahead of print]
Roy G, Weisburg S, Rabindran S, Yusibov V. A novel two-component Tobacco mosaic virus-based vector system for high-level expression of multiple therapeutic proteins including a human monoclonal antibody in plants. Virology. 2010, 405(1):93-9.
Rybicki E P. Plant-made vaccines for humans and animals. Plant Biotechnol 3. 2010, 8(5):620-37
Streatfield S J. Approaches to achieve high-level heterologous protein production in plants. Plant Biotechnol 3. 2007, 5(1):2-15.
Yusibov V, Streatfield S J, Kushnir N. Clinical development of plant-produced recombinant pharmaceuticals: vaccines, antibodies and beyond. Hum Vaccin. 2011, 7(3): 313-21.

1-42. (canceled)

43. An isolated nucleic acid molecule for producing two or more target polypeptides in a plant cell, comprising a minireplicon derived from a Closteroviridae virus and two or more heterologous polynucleotides, wherein the nucleic acid molecule is capable of replicating in the plant cell, and wherein the two or more heterologous polynucleotides encode the two or more target polypeptides.

44. The nucleic acid molecule of claim 43, wherein the Closteroviridae virus is Beet yellows virus.

45. The nucleic acid molecule of claim 43, wherein the plant cell is in a plant, a plant part, or a cell culture medium.

46. The isolated nucleic acid molecule of claim 43, further comprising a polynucleotide encoding one or more movement proteins derived from the Closteroviridae virus.

47. The isolated nucleic acid molecule of claim 43, wherein the two or more target polypeptides comprise two or more subunits of a protein, and wherein the two or more target polypeptides are capable of forming the protein in the plant cell.

48. The isolated nucleic acid molecule of claim 47, wherein the protein is an enzyme.

49. The isolated nucleic acid molecule of claim 43, wherein the two or more target polypeptides comprise a first polypeptide and a second polypeptide, and wherein the first polypeptide is capable of modifying the second polypeptide in the plant cell.

50. The isolated nucleic acid molecule of claim 43, wherein the two or more target polypeptides comprise a first polypeptide and a second polypeptide, and wherein the first polypeptide is capable of affecting expression of the second polypeptide in the plant cell.

51. The isolated nucleic acid molecule of claim 43, wherein the two or more target polypeptides comprise a first polypeptide and a second polypeptide, and wherein the first polypeptide is capable of increasing production of the second polypeptide in the plant cell.

52. The isolated nucleic acid molecule of claim 43, wherein the two or more target polypeptides comprise a heavy chain and a light chain of an antibody, and wherein the two or more target polypeptides are capable of forming the antibody in the plant cell.

53. The isolated nucleic acid molecule of claim 43, wherein the two or more target polypeptides comprise one or more immunogenic polypeptides.

54. The isolated nucleic acid molecule of claim 43, wherein one or more of the target polypeptides comprise a polypeptide of at least 100 kD.

55. A method for producing two or more target polypeptides in a plant cell, comprising
(a) introducing a nucleic acid molecule into the plant cell, wherein the nucleic acid molecule comprises a minireplicon derived from a Closteroviridae virus and two or more heterologous polynucleotides, wherein the nucleic acid molecule is capable of replicating in the plant cell, and wherein the two or more heterologous polynucleotides encode the two or more target polypeptides; and
(b) maintaining the plant cell under conditions permitting production of the two or more target polypeptides in the plant cell.

56. The method of claim 55, further comprising purifying at least one of the two or more target polypeptides from the plant cell.

57. The method of claim 55, wherein the Closteroviridae virus is Beet yellows virus.

58. The method of claim 55, wherein the plant cell is in a plant, a plant part, or a cell culture medium.

59. The method of claim 55, wherein the nucleic acid molecule further comprises a polynucleotide encoding one or more movement proteins derived from the Closteroviridae virus.

60. A composition comprising at least one of the two or more target polypeptides produced by the method of claim 55.

61. A method of treating a subject in need of at least one of the two or more target polypeptides produced by the method of claim 55, comprising administering to the subject an effective amount of a pharmaceutical composition comprising the at least one of the two or more target polypeptides.

62. The method of claim 55, wherein the two or more target polypeptides comprise two or more subunits of a protein, and wherein the maintaining conditions further permit production of the protein in the plant cell.

63. The method of claim 62, wherein the protein is an enzyme.

64. A composition comprising the protein produced by the method of claim 62.

65. The composition of claim 64, wherein the protein is an enzyme.

66. A method of treating a subject in need of the protein produced by the method of claim 62, comprising administering to the subject an effective amount of a pharmaceutical composition comprising the protein.

67. The method of claim 55, wherein the two or more target polypeptides comprise a first polypeptide and a second polypeptide, and wherein the maintaining conditions further permit modifying the second polypeptide by the first polypeptide in the plant cell.

68. The method of claim 55, wherein the two or more target polypeptides comprise a first polypeptide and a second polypeptide, and wherein the maintaining conditions further permit affecting expression of the second polypeptide by the first polypeptide in the plant cell.

69. The method of claim 55, wherein the two or more target polypeptides comprise a first polypeptide and a second polypeptide, and wherein the maintaining conditions further permit increasing production of the second polypeptide by the first polypeptide in the plant cell.

70. A method for inducing a protective immune response against a pathogen in a subject, comprising administering to the subject an effective amount of a pharmaceutical composition comprising at least one of the two or more target polypeptides produced by the method of claim 55, wherein the at least one of the two or more target polypeptides comprises an immunogenic polypeptide derived from the pathogen.
